(19) United States
(12) Patent Application Publication
(10) Pub. No.: US 2021/0087246 A1
(43) Pub. Date: Mar. 25, 2021

(54) MULTIPLE SCLEROSIS ASSOCIATED AUTOANTIGENS, AND USE THEREOF IN THERAPY AND DIAGNOSIS

(71) Applicant: TCER AB, Stockholm (SE)

(72) Inventors: Hans Grönlund, Lidingö (SE); Mattias Bronge, Stockholm (SE)

(21) Appl. No.: 16/498,954

(22) PCT Filed: Mar. 29, 2018
(86) PCT No.: PCT/SE2018/050341

§ 371 Date: Sep. 27, 2019

(30) Foreign Application Priority Data
Mar. 29, 2017 (SE) 1750372-3

(51) Int. Cl. C07K 14/47; A61P 25/28; A61K 39/00; A61K 47/56; A61K 47/69; G01N 33/50; G01N 33/564

(52) U.S. Cl. CPC C07K 14/4713; A61P 25/28; A61K 39/0008; A61K 47/56; A61K 47/6921; A61K 2039/6093; G01N 33/505; G01N 33/564; A61K 2039/6006; A61K 2039/5154; A61K 47/6901

(57) ABSTRACT

A tolerogenic composition for use in a method of treatment for multiple sclerosis (MS) in a MS patient exhibiting T-cell autoreactivity against an endogenous epitope corresponding to a T-cell epitope comprised in the amino-acid sequence of SEQ ID NO: 5, the composition comprising a therapeutic T-cell epitope comprising a sequence of 8 consecutive amino acid residues differing from a sub-sequence of SEQ ID NO: 5 by 0-2 residue substitutions, deletions and/or insertions, or the composition comprising a nucleic acid encoding said therapeutic T-cell epitope. A method for determining the degree of multiple sclerosis (MS) related autoimmunity in a test subject, comprising providing a test sample derived from the test subject comprising viable T-cells; quantitating antigen-specific activation of the T-cells of the test sample in vitro in response to a test antigen comprising a T-cell epitope, wherein said T-cell epitope is as the above therapeutic T-cell epitope above; and comparing the quantitated antigen-specific activation to a relevant reference to determine the degree of MS-related autoimmunity in the test subject.

### TECHNICAL FIELD

The present invention relates to treatment and diagnosis of multiple sclerosis.

### BACKGROUND TO THE INVENTION

Multiple Sclerosis

Multiple sclerosis (MS, ICD 10 code G35) is an immune-mediated chronic disease of the central nervous system (CNS). The current paradigm proposes that it is an autoimmune inflammatory disease which results in demyelination and axonal destruction. Mainly affecting young adults between 20 and 40 years of age, with a total of 2.5 million affected people worldwide, MS is one of the leading causes of disability in young people in the developed world and is responsible for a substantial morbidity among the population as well as high costs for the society due to the care needed.

MS is characterized by infiltration of autoreactive T-cells into the CNS through the blood-brain barrier (BBB) where they get activated presumably by antigen presenting cells (APCs). Subsequently, these autoreactive T-cells cause the typical features of MS, neuroinflammation, demyelination and axonal destruction; creating the characteristic histopathological hallmark—plaques. The focal destruction leads to a wide variety of pathological clinical manifestations involving motor, sensory, visual, and autonomic systems. The inflammation is usually transient and some remyelination occurs in between the inflammatory episodes, resulting in distinct attacks (called relapses) of increased neurological dysfunction followed by episodes of partial recovery. However, over time, the recovery becomes lacking and lasting symptoms accumulate.

T-Cells and Autoantigens in MS

CD4⁺ T-Cells and MS

The main physiological role of the CD4⁺ T-cells is to recognize foreign antigens presented by APCs via the MHC class II molecule and subsequently activate and release cytokines to regulate the immune response. Each CD4⁺ T-cell clone has a specific cell surface expressed T-cell receptor (TCR), sensitive to one specific antigen. CD4⁺ T-cells, often referred to as T helper cells, can be further divided into subsets based on function and the cytokines that they produce. Simplistically, type 1 helper (Th1) T-cells main function is to coordinate the immune response against intracellular pathogens, Th2-cells against parasitic infections and extracellular pathogens and Th17 against fungal and bacterial infections.

As a part of the adaptive immune response, the purpose of the CD4⁺ T-cell and its TCR is to be specific against foreign pathogens. However, since the receptor is generated by random rearrangement of the coding genes, it is possible for T-cells with a TCR specific against self-structures to occur, causing auto-reactivity. Autoreactive T-cells are negatively selected and removed in healthy individuals, but defects in central and peripheral tolerance can give rise to lasting autoreactive T-cells. Such CD4⁺ T-cells are believed to play a central role in the pathogenesis of MS. The fact that certain genes coding for MHC class II have been strongly linked to the disease and the detection of a large number of CD4⁺ T-cells in MS lesions suggests that antigen presentation by APCs and subsequent CD4⁺ T-cell activation plays an imperative part in the disease. Likewise, CD8+T-cells are also present in MS lesions and while their exact role in the disease pathophysiology remains unclear, it is likely they also play a role. The widely-used mouse model experimental autoimmune encephalitis (EAE), which mimics MS, is induced via immunization of mice with myelin-derived peptides, further indicating that auto-reactivity plays a part in MS. Organ-specific autoimmune diseases such as MS have generally been considered to be Th1 mediated, but recent studies have shown that Th17 cells can similarly drive the autoimmune reaction.

Both the T-cells and APCs are present in the fraction of cells commonly named peripheral blood mononuclear cells (PBMCs). PBMCs are a heterogeneous group of cells derived from peripheral blood containing T-cells, B-cells, natural killer cells, monocytes and dendritic cells.

The Cytokines of T-Cells

Upon activation by an APC, the different subsets of CD4⁺ T-cells produce a wide variety of different cytokines. Their function can be to induce proliferation and maturation of other cells or to regulate the overall intensity and duration of inflammation.

Interferon gamma (IFNγ) is a multipotent pro-inflammatory cytokine that is highly expressed by, and acts as the major product of Th1 cells. IFNγpromotes cytotoxic activities of other cells, activates macrophages, regulates expression of MHC class I and II and contribute to further Th1 cell differentiation of naive T-cells. When an APC under certain circumstances activates an antigen specific CD4⁺ T-cell, high amounts of IFNγ are released to drive the T-cell towards Th1 differentiation. Interleukin 17A (IL-17A) is the main cytokine of the CD4⁺ T-cell subgroup Th17 and upregulates the production and secretion of pro-inflammatory cytokines, chemokines and metalloproteases of other cells. IL-17A has been shown to be involved in MS and has been found to be upregulated in mouse models of EAE as well as in patients with other autoimmune diseases such as RA, psoriasis and inflammatory bowel disease. Interleukin 22 (IL-22) is found in activated T-cells, mainly expressed by memory CD4⁺ T-cells, Th17 cells and the recently characterized Th22 cells. It has been found to promote BBB-disruption and CNS inflammation together with IL-17A and is believed to be an important cytokine in the pathogenesis of MS.

Autoantigens in MS

For a CD4⁺ T-cell to become activated, it has to recognize its specific antigen presented by an APC. In the case of autoreactive T-cells, the antigen is a self-protein, so called autoantigen. Several different autoantigens in MS have been proposed and studied (Elong Ngono A, Pettre S, Salou M, Bahbouhi B, Soulillou J P, Brouard S, et al. Frequency of circulating autoreactive T cells committed to myelin determinants in relapsing-remitting multiple sclerosis patients. Clin Immunol. 2012; 144(2):117-26.). The most studied include myelin-, astrocyte-, and neuronal-derived antigens but there have been suggestions of others (Riedhammer C, Weissert R. Antigen Presentation, Autoantigens, and Immune Regulation in Multiple Sclerosis and Other Autoimmune Diseases. Front Immunol. 2015; 6:322). Among studied candidate autoantigens are Myelin Basic Protein (MBP), Myelin Oligodendrocyte Glycoprotein (MOG), Proteolipid Protein (PLP), Myelin Associated Glycoprotein (MAG), Myelin Oligodendrocyte Basic Protein (MOBP), CNPase, S100β and Transaldolase, MBP being the most thoroughly investigated. For these candidates, T-cell auto-reactivity or auto-antibodies have been found in some human studies and animal models. The results however are inconclusive and the data lacks consistency. Despite the difficulty in finding proof, autoantigens and their activation of CD4³⁰ T-cells are still believed to play a key part in the pathogenesis of MS (Hohlfeld R, Dornmair K, Meinl E, Wekerle H. The search for the target antigens of multiple sclerosis, part 1: autoreactive CD4⁺ T lymphocytes as pathogenic effectors and therapeutic targets. Lancet Neurol. 2015).

T-Cell Epitopes

T-cells specific to an antigen do not recognize the whole amino acid (aa) sequence of the antigen, but rather a much shorter specific T-cell epitope contained somewhere in the antigen. The epitopes are typically between 8-11 aa long when presented in an MHC class I molecule and 13-17 aa long when presented in an MCH class II molecule. When an APC internalizes an antigen, it is digested into shorter peptide fragments which are then presented to T-cells via the MHC molecule on the APC surface. These digested fragments of the antigen are the potential specific T-cell epitopes.

Antigen Specific Immunotherapy

Antigen-specific immunotherapy is believed to be a potentially effective future treatment of MS. The goal of the treatment is to induce immune-tolerance, either by depletion of the autoantigen-specific disease-driving T-cells or induction of a favorable immune response (regulatory). Among principal ways to achieve this is either to apply the autoantigen, for example the immunodominant peptide epitopes, in a tolerogenic way via oral, dermal or subcutaneous injection route or to use antigen specific T-cells or their receptor as vaccines to induce a regulatory response. The common theme for the different approaches is the antigen targets used. This treatment strategy has been successful in general terms in that it is well established that T-cell tolerance can be induced via a variety of approaches. Encouraging results have been reported in the mouse model of MS, experimental autoimmune encephalomyelitis (EAE), but so far there has been limited success in human-trials resulting in either no or modest effect. One of the main reasons for this is that the target autoantigens in MS, unlike for EAE, is still not fully known and the optimal targets, or enough targets, might not have been used; “One of the main obstacles in discovering and developing antigen-specific therapies is, of course, our ignorance of the target antigens of multiple sclerosis” (Hohlfeld R, Dornmair K, Meinl E, Wekerle H. The search for the target antigens of multiple sclerosis, part 1: autoreactive CD4⁺ T lymphocytes as pathogenic effectors and therapeutic targets. Lancet Neurol. 2015).

Gap of Knowledge

In summary, the activation of CD4⁺ T-cells by APCs and the autoantigens that they recognize is believed to play a key part in the pathogenesis of MS. The association between MHC class II and MS, the effectiveness of immunomodulating therapies targeting T-cells (e.g. natalizumab), the findings of CD4⁺ T-cells in MS-lesions and the discovery of some autoreactive T-cells in earlier studies all strengthen this hypothesis. However, exactly which autoantigens trigger the autoreactive T-cells and drive the inflammation remain unknown, even though several have been studied. The identification of autoantigens become increasingly important considering the prospect of antigen-specific immunotherapies. The findings so far are however inconclusive and there is a need in the art to identify additional antigens involved in MS pathogenesis.

Thus, an object of the present invention is the provision of improved or alternative means and methods for determining multiple sclerosis -related autoimmunity in a subject, and provision of improved means, methods and compositions for use in the treatment of MS.

### DEFINITIONS

Sequence identity expressed in percentage is defined as the value determined by comparing two optimally aligned sequences over a comparison window, wherein a portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Unless indicated otherwise, the comparison window is the entire length of the sequence being referred to. In this context, optimal alignment is the alignment produced by the BLASTP algorithm as implemented online by the US National Center for Biotechnology Information (see The NCBI Handbook [Internet], Chapter 16), with the following input parameters: Word length=3, Matrix=BLOSUM62, Gap cost=11, Gap extension cost=1.

The term antigen in the context of the present invention refers to a molecule (typically a polypeptide) that contains a specific T-cell epitope.

The term specific T-cell epitope is defined as the part of an antigen that is recognized by T-cells. Typically, specific T-cell epitopes are amino acid sequences between 8-11 aa long when presented in an MHC class I molecule and 13-17 aa long when presented in an MCH class II molecule.

The term MS-antigen refers to an antigen relevant in the pathology of multiple sclerosis (MS).

Endotoxins, e.g. Lipopolysaccharide (LPS), comprise covalently linked lipid and polysaccharide subunits found on the outer cell wall of gram-negative bacteria, such as Escherichia coli.

CD4⁺ T-cell or T-helper cells are cells that orchestrate immune responses through cytokine secretion. They can both suppress or potentiate other immune cells such as stimulate antibody class switching of B-cells, expansion of cytotoxic T-cells or potentiate phagocytes. They get activated by antigen presentation via MHC class II on APCs and they express a T-cell receptor (TCR) specific for a stretch of approximately 13-17 amino acids (a so-called T-cell epitope) within a particular antigen.

CD8⁺ T-cell or cytotoxic T-cells are cells that kill tumour cells, infected cells or cells otherwise damaged. Unlike CD4⁺ T-cells they do not need specialized APCs for activation. Their T-cell receptor recognizes antigen derived peptides (approximately 8-11 amino acids long) presented by MHC class I, a protein expressed on all nucleated cells.

Antigen-specific T-cell activation is a process requiring interaction between the TCR and a defined peptide presented on a MHC (HLA) molecule in combination with co-stimulation.

Antigen-presenting cells (APC) are typically dendritic cells (DCs), B-cells or macrophages, cells that either phagocyte or internalise extra-cellular organisms or proteins, i.e. antigens, and after processing present antigen-derived peptides on MHC class II to CD4⁺ T-cells. In blood, monocytes are the most abundant antigen-presenting cells.

A phagocytable particle is defined as a particle able to be phagocytosed by cells of the immune system, in particular monocytes.

Peripheral blood mononuclear cells (PBMC), is a fraction of human blood prepared by density gradient centrifugation of whole blood. The PBMC fraction mainly consists of lymphocytes (70-90%) and monocytes (10-30%), while red blood cells, granulocytes and plasma have been removed.

Protein epitope signature tag (PrEST) short recombinant 10-12 kDa peptides representing unique parts of human proteins (Lindskog M, Rockberg J, Uhlen M, Sterky F. Selection of protein epitopes for antibody production. Biotechniques. 2005; 38(5):723-7.)

The term peptidomimetic in the context of the present application is defined as a peptide-like polymer chain designed to structurally mimic a peptide but having in some respects different or improved properties.

The term treatment in the present context refers to treatments resulting in a beneficial effect on a subject or patient afflicted with the condition to be treated, including any degree of alleviation, including minor alleviation, substantial alleviation, major alleviation as well as cure. Preferably, the degree of alleviation is at least a minor alleviation. Since MS is a disease with an episodic relapsing character, treatment in the present context also refers to prevention of a relapse or reducing the likelihood of a relapse.

The term prevention in the present context refers to preventive measures resulting in any degree of reduction in the likelihood of developing the condition to be prevented or the condition reoccurring or relapsing, including a minor, substantial or major reduction in likelihood of developing or redeveloping the condition as well as total prevention. Preferably, the degree of likelihood reduction is at least a minor reduction.

### BRIEF DESCRIPTION OF FIGURES

FIG. 1. Autoantigen screening in Multiple Sclerosis-patients against a library of 125 proteins. Comparison of the T-cell activation from MS-patients' PBMCs to that of PBMCs from healthy controls when stimulated with pools of antigens containing PrESTs from 1-2 distinct human proteins. Panel A, activation determination by IFNγ-FluoroSpot. Panel B, activation determination by IL17-FluoroSpot. Panel C, activation determination by IL22-FluoroSpot. Patient's T-cells react significantly more to certain proteins in the library. Open squares and filled circles indicate mean activation in patients' and controls' PBMCs respectively, staples denote CI95% of mean. P-value determined using a two-way ANOVA. Asterisks denote P-value.

FIG. 2. IFNγ/IL-22/IL-17 Fluorospot assay comparing T-cell activation in multiple sclerosis (MS)-patients and healthy controls when stimulated with a suspected autoantigen in MS. Results based on the same data as in FIG. 1, representing each of the new MS-markers contained in this application. 16 patients and 9 controls were included. Panel A, activation when stimulated with antigen pool 18, containing FABP7 (SEQ ID NO:1 and 6) and CYB561D2. Panel B, activation when stimulated with antigen pool 23, containing PROK2 (SEQ ID NO:2) and NOVA2. Panel C, activation when stimulated with antigen pool 26, containing RTN3 (SEQ ID NO:3) and SDK2. Panel D, activation when stimulated with antigen pool 29, containing SNAP91 (SEQ ID NO:4) and SNAP25. P-values determined with Mann-Whitney-U test and written when found. Staples denote CI95% of mean.

FIG. 3. Splitting of the antigens pools from the antigen screening. The antigen pools from the screening experiment for which significant difference in activation between patients and controls were detected were further split up. Each of these pools contained PrESTs from 2 different human proteins, in the figure named #1 and #2. 4 patients which reacted with a high degree of activation in the screening were again tested with the separate antigens in an IFNγ/IL-22/IL-17 Fluorospot. All tests were done in triplicates. P-values determined with student's t-test, comparing the number of activated cells in each well (n=12 per antigen). Staples denote SD. The positive antigens were: 18 #2—FABP7. 23 #2—PROK2. 26 #1—RTN3. 29 #2—SNAP91.

FIG. 4. Reactivity profile among patients. Patterns of activation observed among the patients. Each line connects the data points of one patient. For profile 1a, 1b and 3 the dotted line denotes control mean+2SD. For profile 2, the dotted line denotes the three patients mean IL-22 profile. Only 3 patients reacted significantly to the antigen 26 (RTN3), and only with an activation of IFNγ producing cells, making the results from the previous figures regarding antigen 26 non-optimal for showing these specific patients. Figure however clearly shows a distinct group of patients reacting to this antigen, herein named Profile 2. No pattern like this was identified amongst the healthy controls.

FIG. 5. ROC-curves for the individual antigen, sensitivity and specificity when used as a diagnostic marker. Individually, FABP7 (from pool 18) showed the greatest promise of working as a marker for disease and reaches a sensitivity and specificity of 75 and 85% respectively. A-D shows the ROC-curves for each antigen used in isolation. For FIG. 5E, the number of activated cells to either PROK2 or SNAP91 was used, whichever was higher for each specific individual (same for patients and controls). This way of choosing and analysing only the highest response yielded even better ROC-curves than analysing them individually.

Using an average (of PROK2 and SNAP91) resulted in a similar improved ROC-curve, albeit not as good as using only the highest. FIG. 5F is based on the full length recombinant version of antigen 18, FABP7 isoform 2 (SEQ ID NO: 1).

FIG. 6. IFNγ/IL-22/IL-17 Fluorospot assay comparing T-cell activation in multiple sclerosis (MS)-patients and healthy controls when stimulated with full-length recombinant FABP7 isoform 2. 13 patients (of which 7 consisted of new samplings a year after the first sampling of previous included patients and 6 not previously tested, unfilled squares) and 7 controls (none included in previous tests) were further tested with an in-house produced recombinant version of the FABP7 isoform 2 protein (SEQ ID NO:1) . P-values calculated using Mann-Whitney test. Staples denote mean and CI95%.

FIG. 7. IFNγ/IL-22/IL-17 Fluorospot assay stimulating patient cells with overlapping peptides from autoantigens. Overlapping peptides (15aa long, 10aa overlap) spanning the whole of FABP7 isoform 2 and PROK2 were pooled in pools containing 5-7 peptides each. Cells from 6 MS-patients were tested against these in a FluoroSpot assay. Panel A, activation when stimulated with 6 different pools of overlapping peptides from FABP7 (SEQ ID NO:1). Panel B, activation when stimulated with 3 different pools of overlapping peptides from PROK2 (SEQ ID NO: 2). Detailed peptide information available in table 4. Staples denote CI95%. Negative number of activated T-cells in the graph are due to the average response being lower than the negative control.

FIG. 8. IFNγ/IL-22/IL-17 Fluorospot assay comparing T-cell activation in multiple sclerosis (MS)-patients and healthy controls when stimulated with full-length recombinant antigens (FABP7, PROK2, RTN3, SNAP91). Fifty-two Multiple Sclerosis patients and 24 healthy controls were tested for T-cell activation towards recombinant full-length versions of the autoantigens in a FluoroSpot assay. A: FABP7 isoform 2 (SEQ ID NO:1). B: PROK2 (SEQ ID NO:2). C: RTN3 SEQ ID NO:3. D: SNAP91 SEQ ID NO: 4. P-values calculated using Mann-Whitney test. Staples denote mean and CI95%.

FIG. 9. ROC-curves for the full-length antigens, Sensitivity and specificity when used as a diagnostic marker. Individually, the four antigens perform similarly as diagnostic markers for disease, all reaching a sensitivity of ˜50% with a specificity of 95%. A: FABP7 isoform 2. B: PROK2. C: RTN3. D: SNAP91. E: Using a combination of all four antigens as a diagnostic test achieves higher sensitivity and specificity. Specifically, utilizing the ratio of IL-17 divided by the square root of IFNγ response towards the mean response to the four antigens achieves a sensitivity of 70% with a specificity of >95%.

FIG. 10. Examples of degrees of binding affinity of FABP7 and PROK2 peptides to HLA DRB5 01:01. Overlapping peptides spanning the whole of FABP7 (SEQ ID NO: 1 and 6) and PROK2 (SEQ ID NO:2) were tested in a competitive binding assay for their affinity towards the multiple sclerosis associated HLA DRB5 01:01. Degree of affinity was divided into four categories and representative binding curves are presented: A: − no binding, B: + weak binding, C: ++ moderate binding, D: +++ strong binding. Black dots denote reference H1N1 influenza peptide binding, triangels the tested autoantigen peptide. Full results in Table 4.

### SUMMARY OF THE INVENTION

The present invention is based on findings from screenings performed on a T-cell reactivity platform previously disclosed by the inventors in PCT/EP2016/081141.

Samples of T-cells from multiple sclerosis (MS)-patients and controls were screened against a library of 125 candidate antigens in 45 pools (Example 1). The positive antigen pools 18, 23, 26 and 29 were split into the individual proteins and re-tested, allowing identification of FABP7 (SEQ ID NO: 1), PROK2 (SEQ ID NO: 2), RTN3 (SEQ ID NO: 3) and SNAP91 (SEQ ID NO: 4) as novel autoantigens in MS (Example 2). It was also discovered that individual patients displayed several different types of reactivity profiles to the novel autoantigens (Example 3).

The diagnostic utility of the novel autoantigens was demonstrated by receiver-operator-characteristic (ROC)-analysis (Example 4). Of the individual antigens, the sensitivity and specificity (always a trade-off) were most promising for FABP7 showing 75% sensitivity and 85% specificity. The sensitivity and specificity were most promising for a combination of all four antigens, with a 70% sensitivity and >95% specificity.

The autoantigens were further validated by testing full-length recombinant versions of each antigen (Example 7), and the principle of identification of the specific T-cell epitope was demonstrated using overlapping peptides (15 aa, 10 aa overlap) covering the entire sequence of FABP7 and PROK2 (Example 6). Further, the HLA-binding properties of the different peptide-epitopes was demonstrated (Example 6).

Given that the prior art has demonstrated that it is possible to successfully treat MS with a tolerogenic composition comprising disease-related T-cell epitopes, the inventors realized that the discovery of the autoantigens further provides a new treatment for MS (see Example 8).

As discussed in the Background section, methods for inducing T-cell tolerance are known, but the treatment of MS has been hampered by lack of suitable antigens to which T-cell tolerance can be induced. The biological mechanism of the inventive treatment of MS is based on inducing antigen-specific T-cell tolerance, which is well accepted in the field.

Although the initial academic studies were based on the concept of four novel autoantigens as separate entities (due to their natural biological context), it was subsequently realized that given the short nature of the T-cell epitopes used both in diagnosis and therapy and the fact that combining all four autoantigens improved the diagnostic results (ROC analysis), it is both possible and advantageous to consider the pooled sequences of all four novel autoantigens as a single source of specific T-cell epitopes (SEQ ID NO: 5) for use in the invention. The sequence of SEQ ID NO: 5 is a combination of the sequences of FABP7 isoform 2 (SEQ ID NO: 1), PROK2 (SEQ ID NO: 2), RTN3 (SEQ ID NO: 3), SNAP91 (SEQ ID NO: 4) and the unique part of FABP7 isoform 1 (SEQ ID NO: 6).

The present invention relates specifically to the following items. The subject matter disclosed in the items below should be regarded disclosed in the same manner as if the subject matter were disclosed in patent claims.
1. A tolerogenic composition for use in a method of treatment for multiple sclerosis (MS) in a MS patient exhibiting T-cell autoreactivity against an endogenous epitope corresponding to a specific T-cell epitope comprised in the amino-acid sequence of SEQ ID NO: 5,
   the composition comprising a therapeutic T-cell epitope comprising a sequence of n consecutive amino acid residues being:
      a. identical to a sub-sequence of SEQ ID NO: 5; or
      b. differing from a sub-sequence of SEQ ID NO: 5 by no more than m residue substitutions, deletions and/or insertions;
      wherein n is at least 8, and m is 0, 1 or 2.
2. A tolerogenic composition for use in a method of treatment for multiple sclerosis (MS) in a MS patient exhibiting T-cell autoreactivity against an endogenous epitope corresponding to a specific T-cell epitope comprised in the amino-acid sequence of SEQ ID NO: 5,
   the composition comprising a nucleic acid encoding a therapeutic T-cell epitope comprising a sequence of n consecutive amino acid residues being:
      a. identical to a sub-sequence of SEQ ID NO: 5; or
      b. differing from a sub-sequence of SEQ ID NO: 5 by no more than m residue substitutions, deletions and/or insertions;
      wherein n is at least 8, and m is 0, 1 or 2.
3. A tolerogenic composition for use in a method of treatment for multiple sclerosis (MS) in a MS patient exhibiting T-cell autoreactivity against an endogenous epitope corresponding to a specific T-cell epitope comprised in the amino-acid sequence of SEQ ID NO: 5,
   the composition comprising an antigen-presenting cell exposed ex vivo to a therapeutic T-cell epitope comprising a sequence of n consecutive amino acid residues being:
      a. identical to a sub-sequence of SEQ ID NO: 5; or
      b. differing from a sub-sequence of SEQ ID NO: 5 by no more than m residue substitutions, deletions and/or insertions;
      wherein n is at least 8, and m is 0, 1 or 2.
4. The composition for use according to any of the preceding items, the method of treatment comprising determining the patient's T-cell autoreactivity against a T-cell epitope comprised in the amino-acid sequence of SEQ ID NO: 5.
5. The composition for use according to item 4, wherein the determining is performed with a method according to any of items 43-110.
6. The composition for use according to any of items 1-5, wherein the sub-sequence is comprised in residues 1-166 of SEQ ID NO: 5.
7. The composition for use according to any of items 1-5, wherein the sub-sequence is comprised in residues 167-295 of SEQ ID NO: 5.
8. The composition for use according to any of items 1-5, wherein the sub-sequence is comprised in residues 296-1327 of SEQ ID NO: 5.
9. The composition for use according to any of items 1-5, wherein the sub-sequence is comprised in residues 1328-2234 of SEQ ID NO: 5.
10. The composition for use according to any of items 1-5, wherein the sub-sequence is comprised in residues 2235-2250 of SEQ ID NO: 5.
11. The composition for use according to any of items 1-5, wherein the sub-sequence is comprised in residues 1-2234 of SEQ ID NO: 5.
12. The composition for use according to any of items 1-5, wherein the sub-sequence is comprised in any one of the sequences of peptides in table 4.
13. The composition for use according to any of the preceding items, wherein n is at least 11.
14. The composition for use according to any of the preceding items, wherein n is at least 13.
15. The composition for use according to any of the preceding items, wherein n is at least 15.
16. The composition for use according to any of the preceding items, wherein n is at least 17.
17. The composition for use according to any of the preceding items, wherein n is at least 19.
18. The composition for use according to any of the preceding items, wherein n is at least 50, at least 75, most preferably at least 100.
19. The composition for use according to any of the preceding items, wherein m is 2.
20. The composition for use according to any of items 1-18, wherein m is 1.
21. The composition for use according to any of items 1-18, wherein m is 0.
22. The composition for use according to any of the preceding items, wherein said T-cell epitope differs from a sub-sequence by no more than m residue substitutions, and comprises no substitutions or deletions compared to the sub-sequence.
23. The composition for use according to item 1 or 3, or any item dependent thereon, wherein the therapeutic T-cell epitope is a peptide or a peptidomimetic.
24. The composition for use according item 23, wherein the therapeutic T-cell epitope is a peptide or peptidomimetic having at least 80%, preferably at least 90%, more preferably at least 95%, most preferably at least 98% sequence identity to a subsequence of SEQ ID NO: 5.
25. The composition for use according to item 1 or any item dependent thereon, wherein the composition comprises 2, 3, 4, 5, 6, 7, 8, 9, 10 or more different therapeutic T-cell epitopes each fulfilling the criteria set out in item 1.
26. The composition for use according to item 2 or any item dependent thereon, wherein the composition comprises a nucleic acid encoding 2, 3, 4, 5, 6, 7, 8, 9, 10 or more different therapeutic T-cell epitopes each fulfilling the criteria set out in item 2.
27. The composition for use according to item 3 or any item dependent thereon, wherein the antigen-presenting cells have been exposed to 2, 3, 4, 5, 6, 7, 8, 9, 10 or more different therapeutic T-cell epitopes each fulfilling the criteria set out in item 3.
28. The composition for use according to any of items 25-27, wherein the different therapeutic T-cell epitopes are selected from one, two, three, four, five or six of the sequence intervals specified in items 6-11.
29. The composition for use according to item 1 or any item dependent thereon, wherein composition comprises the therapeutic T-cell epitope coupled to solid carrier, such as a biocompatible polymer, a particle or a cell.
30. The composition for use according to item 29, wherein the coupling is via a covalent bond.
31. The composition for use according to any of the preceding items, the composition being formulated for inducing T-cell tolerance towards said therapeutic T-cell epitope the patient.
32. The composition for use according to item 1 or any item dependent thereon, the composition comprising a therapeutic T-cell epitope capable of specifically binding to the same T-cell receptor as said endogenous epitope.
33. The composition for use according to item 2 or any item dependent thereon, the composition comprising a nucleic acid encoding a therapeutic T-cell epitope capable of specifically binding to the same T-cell receptor as said endogenous epitope.
34. The composition for use according to item 3 or any item dependent thereon, the composition comprising antigen-presenting cells exposed to a therapeutic T-cell epitope capable of specifically binding to the same T-cell receptor as said endogenous epitope.
35. The composition for use according to any of the preceding items, the method comprising selecting the therapeutic T-cell epitope such that it corresponds to an epitope to which the patient exhibits T-cell autoreactivity.
36. The composition for use according to item 35, wherein the therapeutic T-cell epitope is selected based on it being able to specifically bind to the same TCR as said endogenous epitope.
37. The composition for use according to any of the preceding items, the method of treatment comprising administering the composition in a tolerogenic manner to the subject thus inducing T-cell tolerance towards said T-cell epitope in the patient.
38. The composition for use according to any of the preceding items, the method of treatment comprising administering the composition orally, mucosally, intradermally, transdermally or subcutaneously.
39. The composition for use according to item 1 or 2, or any item dependent thereon, the method of treatment comprising administering the composition in vitro to antigen-presenting cells, followed by administering said antigen-presenting cells to the patient.
40. The composition for use according to item 2 or any item dependent thereon, wherein said nucleic acid is included in a vector, operatively coupled to a promoter allowing expression in cells, preferably antigen-presenting cells.
41. The composition for use according to item 40, wherein said vector is a gene therapy vector or a viral vector.
42. The composition for use according to item 3 or any item dependent thereon, wherein the antigen-presenting cells are dendritic cells, monocytes, macrophages, B-cells (preferably derived from PBMCs) or microglia.
43. A method for determining the degree of multiple sclerosis (MS) related autoimmunity in a test subject, comprising:
   a. providing a test sample derived from the test subject comprising viable T-cells;
   b. quantitating antigen-specific activation of the T-cells of the test sample in vitro in response to a test antigen comprising a T-cell epitope, wherein said T-cell epitope comprises an amino-acid sequence of n consecutive residues being:
      i. identical to a sub-sequence of SEQ ID NO: 5; or
      ii. differing from a sub-sequence of SEQ ID NO: 5 by no more than m residue substitutions, deletions and/or insertions;
      wherein n is at least 8, and m is 0, 1 or 2; and
   c. comparing the quantitated antigen-specific activation to a relevant reference to determine the degree of MS-related autoimmunity in the test subject.
44. The method according to item 43, wherein the sub-sequence is comprised in residues 1-166 of SEQ ID NO: 5.
45. The method according to item 43, wherein the sub-sequence is comprised in residues 167-295 of SEQ ID NO: 5.
46. The method according to item 43, wherein the sub-sequence is comprised in residues 296-1327 of SEQ ID NO: 5.
47. The method according to item 43, wherein the sub-sequence is comprised in residues 1328-2234 of SEQ ID NO: 5.
48. The method according to item 43, wherein the sub-sequence is comprised in residues 2235-2250 of SEQ ID NO: 5.
49. The method according to item 43, wherein the sub-sequence is comprised in residues 1-2234 of SEQ ID NO: 5.
50. The method according to item 43, wherein the sub-sequence is comprised in any one of the sequences of peptides in table 4.
51. The method according to any of the preceding method items, wherein antigen-specific activation is quantitated against at 2, 3, 4, 5, 6, 7, 8, 9, 10 or more different T-cell epitopes each fulfilling the criteria set out in item 43.
52. The method according to item 51, wherein the different T-cell epitopes are selected from one, two, three, four, five or six of the sequence intervals specified in items 44-49.
53. The method according to any of the preceding method items, wherein antigen-specific activation is quantitated against at least two, three or four test antigens each comprising a specific T-cell epitope corresponding to a different MS-antigen selected from the group consisting of FABP7 (SEQ ID NO: 1), PROK2 (SEQ ID NO: 2), RTN3 (SEQ ID NO: 3) and SNAP91 (SEQ ID NO: 4).
54. The method according to any of the preceding method items, wherein said sub-sequence is included in the sequence of FABP7 (SEQ ID NO: 1) residues 1-82.
55. The method according to any of the preceding method items, wherein said sub-sequence is included in the sequence of FABP7 (SEQ ID NO: 1) residues 83-166.
56. The method according to any of the preceding method items, wherein said sub-sequence is included in the sequence of FABP7 (SEQ ID NO: 1) residues 105-166.
57. The method according to any of the preceding method items, wherein said sub-sequence is included in the sequence of PROK2 (SEQ ID NO: 2) residues 34-74.
58. The method according to any of the preceding method items, wherein said sub-sequence is included in the sequence of PROK2 (SEQ ID NO: 2) residues 106-128.
59. The method according to any of the preceding method items, wherein said sub-sequence is included in the sequence of RTN3 (SEQ ID NO: 3) residues 81-217.
60. The method according to any of the preceding method items, wherein said sub-sequence is included in the sequence of RTN3 (SEQ ID NO: 3) residues 345-483.
61. The method according to any of the preceding method items, wherein said sub-sequence is included in the sequence of SNAP91 (SEQ ID NO: 4) residues 378-480.
62. The method according to any of the preceding method items, wherein said sub-sequence is included in the sequence of SNAP91 (SEQ ID NO: 4) residues 481-572.
63. The method according to any of the preceding method items, wherein said sub-sequence is included in the sequence of SNAP91 (SEQ ID NO: 4) residues 584-691.
64. The method according to any of the preceding method items, wherein n is at least 11.
65. The method according to any of the preceding method items, wherein n is at least 13.
66. The method according to any of the preceding method items, wherein n is at least 15.
67. The method according to any of the preceding method items, wherein n is at least 17.
68. The method according to any of the preceding method items, wherein n is at least 19.
69. The method according to any of the preceding method items, wherein n is at least 50, at least 75, most preferably at least 100.
70. The method according to any of the preceding method items, wherein m is 2.
71. The method according to any of items 43-69, wherein m is 1.
72. The method according to any of items 43-69, wherein m is 0.
73. The method according to any of the preceding method items, wherein said T-cell epitope differs from a sub-sequence of the selected MS-antigen by no more than m residue substitutions, and comprises no substitutions or deletions compared to the sub-sequence.
74. The method according to any of the preceding method items, wherein said T-cell epitope is a peptide or peptidomimetic.
75. The method according to any of the preceding method items, wherein the antigen comprising a specific T-cell epitope is a peptide or peptidomimetic having at least 80%, preferably at least 90%, more preferably at least 95%, most preferably at least 98% sequence identity to any of SEQ ID NOs: 1-4, SEQ ID NO: 5 or SEQ ID NO: 6.
76. The method according to any of the preceding method items, wherein the subject is a diagnosed MS-patient.
77. The method according to any of items 43-75, wherein the subject is an individual suspected of having MS.
78. The method according to any of the preceding method items, wherein the subject is a human.
79. The method according to any of the preceding method items, wherein the test sample is derived from a blood sample.
80. The method according to any of the preceding method items, wherein the test sample is a PBMC sample.
81. The method according to any of the preceding method items, wherein the test sample further comprises antigen-presenting cells.
82. The method according to any of the preceding method items, wherein the method further comprises:
   a. Providing a viable antigen-presenting cell;
   b. Contacting the test antigen with the antigen-presenting cell;
   c. Contacting in vitro the test sample with the antigen-presenting cell contacted with the test antigen under conditions allowing antigen-specific activation of T-cells in response to an antigen presented by an antigen-presenting cell; and
   d. Quantitating antigen-specific T-cell activation in the test sample.
83. The method according to any of the preceding method items, wherein the method comprises providing the test antigen tightly associated to a phagocytable particle.
84. The method according to item 83, wherein the method further comprises the steps (a′) tightly associating the test antigen to a phagocytable particle and/or (a″) subjecting the test antigen associated with a particle to a denaturing wash resulting in an endotoxin level low enough to not interfere with the subsequent steps.
85. The method according to any of the items 83-84, wherein the particle has a largest dimension of less than 5.6 μm, preferably less than 4 μm, more preferably less than 3 μm, even more preferably in the interval 0.5-2 μm or most preferably about 1 μm.
86. The method according to item 85, wherein the particle is substantially spherical.
87. The method according to any items 84-86 wherein the denaturing wash involves subjecting the particle with the associated test antigen to a high pH, such as at least pH 13, more preferably at least pH 14, most preferably at least pH 14.3.
88. The method according to any of items 84-87, wherein the denaturing wash involves subjecting the particle with the associated test antigen to a low pH.
89. The method according to any of items 84-88, wherein the denaturing wash involves subjecting the particle with the associated test antigen to a high temperature, such as at least 90° C., more preferably at least 92° C., most preferably at least 95° C.
90. The method according to any of items 84-89, wherein the denaturing wash involves subjecting the particle with the associated test antigen to a denaturing agent, such as urea or guanidine hydrochloride at a sufficient concentration, such as at least 5M, 6M, 7M or 8M.
91. The method according to any of items 84-90, wherein the denaturing wash results in an endotoxin amount in the test antigen being such that in the method, the final concentration of endotoxin is less than 100 pg/ml, preferably less than 50 pg/ml, more preferably less than 25 pg/ml and most preferably less than 10 pg/ml.
92. The method according to any of items 83-91, wherein the particle has paramagnetic properties.
93. The method according to any of items 83-92, wherein the test antigen is covalently linked to the particle or linked to the particle via a metal chelate.
94. The method according to any of the preceding method items, wherein quantitating the antigen-specific T-cell activation in the test sample is performed using an ELISpot or a FluoroSpot-technique, or by measuring T-cell proliferation.
95. The method according to any of the preceding method items, wherein quantitating the antigen-specific T-cell activation in the test sample comprises determining the T-cell response by measuring secretion of IFN-γ.
96. The method according to any of the preceding method items, wherein quantitating the antigen-specific T-cell activation in the test sample comprises determining the T-cell response by measuring secretion of IL-17.
97. The method according to any of the preceding method items, wherein quantitating the antigen-specific T-cell activation in the test sample comprises determining the T-cell response by measuring secretion of IL-22.
98. The method according to any of the preceding method items, wherein quantitating T-cell activation in the test cell sample involves determining the fraction of activated T-cells in the sample.
99. The method according to any of the preceding method items, wherein the quantitation of antigen-specific T-cell activation comprises the steps of:
   a. Providing a phagocytable particle, having the test antigen tightly associated thereto, wherein the particle with the associated test antigen has been subjected to a denaturing wash resulting in an endotoxin level low enough to not interfere with the subsequent steps;
   b. Providing a viable antigen-presenting cell;
   c. Contacting the washed particle with the antigen-presenting cell under conditions allowing phagocytosis of the particle by the antigen-presenting cell;
   d. Providing the test sample to be assayed comprising viable T-cells;
   e. Contacting in vitro the test sample with the antigen-presenting cell contacted with the particle under conditions allowing antigen-specific activation of T-cells in response to an antigen presented by an antigen-presenting cell; and
   f. Quantitating antigen-specific T-cell activation in the test sample.
100. The method according to any of the preceding method items, wherein the reference is comparably quantitated antigen-specific activation in a reference sample from a reference subject free of pathological MS-related autoimmunity.
101. The method according to any of any of the items 43-99, wherein the reference is a mean value of comparably quantitated antigen-specific activation in a set of reference samples from a set of reference subjects free of pathological MS-related autoimmunity.
102. The method according to item 101, wherein the set comprises at least 10 reference subjects.
103. The method according to any of the items 43-99, wherein the reference is comparably quantitated antigen-specific activation in a sample from the same subject taken at a different point in time.
104. The method according to any of the preceding method items, wherein an increased degree of MS-related autoimmunity is concluded if the quantitated antigen-specific activation in the test sample is at least 2 times, preferably 3 times, more preferably 5 times, most preferably 10 times higher compared to the reference.
105. The method according to any of the preceding method items, wherein an increased degree of MS-related autoimmunity is concluded if the quantitated antigen-specific activation in the test sample is higher than the mean of a set of comparably quantitated reference samples from a set of reference subjects free of pathological MS-related autoimmunity by 2 times the standard deviation of the set of reference samples.
106. The method according to any of the preceding method items, wherein an increased degree of MS-related autoimmunity is concluded if the quantitated antigen-specific activation in the test sample is statistically significantly higher than the reference with a p value of less than 0.05 calculated with Student's T-test.
107. The method according to any of the preceding method items, wherein an increased degree of MS-related autoimmunity is concluded if the quantitated antigen-specific activation in the test sample is statistically significantly higher than the reference with a p value of less than 0.05 calculated with Mann-Whitney U-test.
108. The method according to any of the preceding method items, wherein:
   a. the method is for diagnosing MS in the subject;
   b. the reference is representative of comparably quantitated antigen-specific activation in reference subjects free of pathological multiple sclerosis related autoimmunity; and
   c. a higher degree of quantitated antigen-specific activation in the test sample compared to the reference is indicative of multiple sclerosis in the test subject.
109. The method according to any of the preceding method items, wherein:
   a. the method is for following course of MS in the subject;
   b. the reference is representative of comparably quantitated antigen-specific activation in a sample taken at a different point in time from the same subject; and
   c. a higher degree of quantitated antigen-specific activation in the test sample compared to the reference is indicative of higher multiple sclerosis disease activity in the test subject, and vice versa.
110. The method according to any of the preceding method items, wherein:
   a. the method is for making prognosis of MS course in the subject;
   b. the reference is representative of comparably quantitated antigen-specific activation in a sample taken at a different point in time from the same subject; and
   c. a higher degree of quantitated antigen-specific activation in the test sample compared to the reference is indicative of increasing multiple sclerosis disease activity in the test subject, and vice versa.
111. The method according to any of the preceding method items, wherein:
   a. the method is for evaluating response of the subject to a therapeutic treatment;
   b. the reference is representative of comparably quantitated antigen-specific activation in a sample taken from the same subject prior to administration of the therapeutic treatment to be evaluated;
   c. the test sample is from the same subject after initiation of the therapeutic treatment; and
   d. a lower degree of quantitated antigen-specific activation in the test sample compared to the reference is indicative of therapeutic efficacy against multiple sclerosis disease activity in the test subject, and unchanged or higher degree of quantitated antigen-specific activation is indicative of lack of therapeutic efficacy against multiple sclerosis disease activity in the test subject.
112. An use of a peptide or peptidomimetic in the diagnosis or treatment of MS, wherein the peptide or peptidomimetic comprises a specific T-cell epitope corresponding to a MS-antigen, said T-cell epitope comprising an amino-acid sequence of n consecutive residues differing from a sub-sequence of SEQ ID NO: 5 by no more m residue substitutions, deletions and/or insertions, wherein m is 0, 1 or 2.
113. The use according to item 112, wherein the sub-sequence is selected from residues 1-166 of SEQ ID NO:5, residues 167-295 of SEQ ID NO:5, residues 296-1327 of SEQ ID NO:5, residues 1328-2234 of SEQ ID NO:5, residues 2235-2250 of SEQ ID NO: 5 and/or residues 1-2234 of SEQ ID NO: 5.
114. The use according to item 112 or 113, wherein n is at least 11.
115. The use according to item 112 or 113, wherein n is at least 13.
116. The use according to item 112 or 113, wherein n is at least 15.
117. The use according to item 112 or 113, wherein n is at least 17.
118. The use according to item 112 or 113, wherein n is at least 19.
119. The use according to any of items 112-118, wherein m is 1.
120. The use according to any of items 112-118, wherein m is 0.

### DETAILED DESCRIPTION

Tolerogenic compositions for use in the treatment of multiple sclerosis In a first aspect, the present invention provided a tolerogenic composition for use in a method of treatment for multiple sclerosis (MS) in a MS patient exhibiting T-cell autoreactivity against an endogenous epitope corresponding to a specific T-cell epitope comprised in the amino-acid sequence of SEQ ID NO: 5,

the composition comprising a therapeutic T-cell epitope comprising a sequence of n consecutive amino acid residues being:
a. identical to a sub-sequence of SEQ ID NO: 5; or
b. differing from a sub-sequence of SEQ ID NO: 5 by no more than m residue substitutions, deletions and/or insertions;
wherein n is at least 8, and m is 0, 1 or 2.

In a second aspect, there is provided a tolerogenic composition for use in a method of treatment for multiple sclerosis (MS) in a MS patient exhibiting T-cell autoreactivity against an endogenous epitope corresponding to a specific T-cell epitope comprised in the amino-acid sequence of SEQ ID NO: 5, the composition comprising a nucleic acid encoding a therapeutic T-cell epitope being as defined for the first aspect.

In a third aspect, there is provided a tolerogenic composition for use in a method of treatment for multiple sclerosis (MS) in a MS patient exhibiting T-cell autoreactivity against an endogenous epitope corresponding to a specific T-cell epitope comprised in the amino-acid sequence of SEQ ID NO: 5, the composition comprising an antigen-presenting cell exposed ex vivo to a therapeutic T-cell epitope being as defined for the first aspect.

It is to be understood as implied that the method of treatment comprises administering the composition to the patient.

The method of treatment may comprise the step of determining the patient's T-cell autoreactivity against a specific T-cell epitope comprised in the amino-acid sequence of SEQ ID NO: 5, prior to administering the composition, preferably by way of the method disclosed below as the fourth aspect of the present invention.

It should be noted that the therapeutic T-cell epitope can be included as part of practically any larger polypeptide (e.g. by way of genetic engineering or chemical peptide synthesis) given that the antigen-presenting cells (APC) of the patient will digest the larger and present the fragments to the T-cells. In other words, due to the digestion by the APCs, the sequence context in which a therapeutic T-cell epitope is found makes little or no difference.

It is preferable that the composition of the first aspect comprises more than one therapeutic T-cell epitopes, such as 2, 3, 4, 5, 6, 7, 8, 9, 10, 12, 14, 16, 18, 20, 30, 40, 50, 100 or more, each fulfilling the criteria set forth above. As discussed above and in the Background section, antigen presenting cells will digest any proteins destined for antigen presentation into small fragments, so it is possible or even preferable to include one or more therapeutic T-cell epitopes in a larger polypeptide or peptidomimetic to be used within the same therapeutic paradigm. To avoid any uncertainty, it is clear that the therapeutic T-cell epitopes may also be separate chemical entities.

The same applies to the composition of the second aspect, with the modification that the composition comprises one or more nucleic acids encoding for more than one therapeutic T-cell epitopes, such as 2, 3, 4, 5, 6, 7, 8, 9, 10, 12, 14, 16, 18, 20, 30, 40, 50, 100 or more, each epitope fulfilling the criteria set forth above. A nucleic acid may encode for more than one therapeutic T-cell epitope, since the cells digest any expressed protein destined for antigen display into small fragments as discussed above. Alternatively, or additionally, it is possible to include the therapeutic T-cell epitopes as part of a longer sequence comprising sequences that are not specific T-cell epitopes, for the aforementioned reasons.

The antigen-presenting cells of the third composition may have been exposed to more than one therapeutic T-cell epitopes, such as 2, 3, 4, 5, 6, 7, 8, 9, 10, 12, 14, 16, 18, 20, 30, 40, 50, 100 or more, each fulfilling the criteria set forth above. As previously discussed, the T-cell epitopes may be included in a larger polypeptide or polypeptides even in this case.

It should be understood that “exposing” in the context of the third aspect may refer not only to contacting the antigen-presenting cells with a peptide or peptidomimetic comprising the therapeutic T-cell epitope, but also to transfecting the antigen-presenting cells with a nucleic acid encoding to the therapeutic T-cell epitope, causing the cells to express the therapeutic T-cell epitope thus exposing the cells.

Sub-Sequence

The sub-sequence of the first, second or third aspects may be included in i) residues 1-166 of SEQ ID NO: 5, ii) residues 167-295 of SEQ ID NO: 5, iii) residues 296-1327 of SEQ ID NO: 5, iv) residues 1328-2234 of SEQ ID NO: 5 or v) in residues 2235-2250 of SEQ ID NO: 5. Preferably, the sub-sequence is included in vi) residues 1-2234 of SEQ ID NO: 5.

The composition of the first aspect may comprise more than one different therapeutic T-cell epitopes as defined above. Preferably, the more than one different therapeutic T-cell epitopes are selected from two, three, four, five or six of the distinct intervals presented above as i)-vi).

The same principle applies to the nucleic acid of the second aspect, mutatis mutandis. Preferably, the nucleic acid encodes for more than one different therapeutic T-cell epitopes as defined above. Preferably, the more than one different encoded T-cell epitopes are selected from two, three, four, five or six of the distinct intervals presented above as i)-vi).

Likewise, the cells of the third aspect may have been exposed to more than one different therapeutic T-cell epitopes as defined above. Preferably, the more than one different therapeutic T-cell epitopes are selected from two, three, four, five or six of the distinct intervals presented above as i)-vi).

The sub-sequence may be comprised in any one of the sequences of peptides in table 4. Preferably, the sub-sequence is comprised in any one of the sequences of peptides in table 4 where the HLA binding is indicated as “++” or “+++”, most preferably “+++”.

Preferably, n is at least 9, 10, 11, 12, 13, 14, 15, 16, 17, 18, 19, 20, 21 or 22. n may be at least 50, at least 75, even more preferably at least 100. n may be any interval formed from the aforementioned numbers, e.g. 8-22, 9-21, 8-17, 8-15, 8-12, or 8-100. Most preferably n is 8-19, or 8-17 when m=0. Preferably, m is 2, more preferably m is 1, most preferably m is 0.

Preferably, the therapeutic T-cell epitope is identical to the sub-sequence (m=0). However, it is probably in most cases possible to vary the sequence slightly and still have a functionally fully or substantially equivalent therapeutic T-cell epitope compared to one having sequence identify to a sub-sequence of SEQ ID NO: 5. For instance, substituting an amino-acid for another with similar characteristics in terms of polarity, charge or hydrophobicity may be tolerable. Small insertions or deletions within the therapeutic T-cell epitope may also be tolerable provided that they result in a functionally fully or substantially equivalent therapeutic T-cell epitope compared to one having sequence identify to a sub-sequence of SEQ ID NO: 5. Preferably, m is 2, more preferably m is 1, most preferably m is 0.

Preferably, said therapeutic T-cell epitope may differ from a sub-sequence by no more than m residue substitutions, and comprises no substitutions or deletions compared to the sub-sequence.

The therapeutic T-cell epitope of the first or the third aspects may be a peptide or a peptidomimetic. The therapeutic T-cell epitope of the first or the third aspects may be peptide or peptidomimetic having at least 80%, preferably at least 90%, more preferably at least 95%, most preferably at least 98% sequence identity to a subsequence of SEQ ID NO: 5.

The nucleic acid of the second aspect may be DNA, PNA or any other nucleic acid capable of encoding a protein for expression in mammalian cells.

Composition Formulations

The composition is formulated for inducing T-cell tolerance towards said therapeutic T-cell epitope the patient, which may be human or a non-human mammal, preferably human.

The composition of the first aspect may comprise the therapeutic T-cell epitope coupled to solid carrier, such as a biocompatible polymer (such as PLGA), a liposome, a solid particle or a cell.

The coupling is preferably via a covalent bond, but other couplings are also possible including metal chelate binding, hydrophobic interactions or ionic interactions. A suitable coupling protocol for coupling antigen peptides to cells is disclosed in EP2205273. A suitable coupling protocol for coupling antigenic peptides to PLGA-microparticles is disclosed by Gholamzad and coworkers (Iranian Journal of Allergy, Asthma and Immunology 2017. 16(3):271-281). A suitable protocol for coupling antigen peptides to a polymeric carrier is disclosed in Pearson and coworkers (Mol Ther. 2017 Jul. 5; 25(7):1655-1664. doi: 10.1016/j.ymthe.2017.04.015).

The composition according to the first aspect may comprise a therapeutic T-cell epitope capable of specifically binding to the same T-cell receptor as said endogenous epitope. By specific binding in the context of the present invention is meant binding that is high enough to lead to T-cell activation in a biological setting in vitro or in vivo.

The composition according to the second aspect may comprise nucleic acid encoding a therapeutic T-cell epitope capable of specifically binding to the same T-cell receptor as said endogenous epitope.

The antigen-presenting cells of the third aspect may have been exposed to a therapeutic T-cell epitope capable of specifically binding to the same T-cell receptor as said endogenous epitope.

Selecting the Therapeutic T-Cell Epitope

The method of treatment of the first, second or third aspects may comprise selecting the therapeutic T-cell epitope such that it corresponds to an endogenous T-cell epitope to which the patient exhibits T-cell autoreactivity.

The therapeutic T-cell epitope may be selected based on it being able to specifically bind to the same TCR as said endogenous epitope.

The therapeutic T-cell epitope(s) of the first, second or third compositions may be individually tailored or selected to match the autoreactivity in the individual patient or subject to be treated. In many cases however, it may be more practicable to administer a composition comprising several therapeutic T-cell epitopes corresponding to the most common endogenous T-cell epitopes, since tailoring a composition is time-consuming, costly and may present regulatory challenges in many jurisdictions.

Administering the Composition

The method of treatment of the first to third aspects preferably comprises administering the composition in a tolerogenic manner to the subject thus inducing T-cell tolerance towards the therapeutic T-cell epitope the patient.

The method of treatment may comprise administering the composition orally, mucosally, intradermally, transdermally or subcutaneously. The administering may be by injection.

The method of treatment of the first or second aspects may comprise administering the composition ex vivo to antigen-presenting cells, followed by administering said antigen-presenting cells to the subject.

The treatment-dose is preferably titrated from a low dose to a higher dose under a period of several weeks/months. The treatment is preferably started with a low first dose, followed by increasing doses (for example doubling) each subsequent administration. After this titration period of several weeks/months a maintenance-level which can be 10-100 times higher than the starting dose can be reached and maintained for a period of time.

The literature describes several protocols for inducing T-cell tolerance in MS and other conditions, which can be adapted for use with the present invention, simply by replacing the T-cell antigen to which tolerance is being induced to a therapeutic T-cell epitope described herein.

Cathaway and co-workers (Cathaway J, Martin K, Barrell K, Sharrack B, Stolt P, Wraith D C. Effects of ATX-MS-1467 immunotherapy over 16 weeks in relapsing multiple sclerosis. Neurology 2018) have conducted an open label study to assess safety, tolerability, and efficacy of an antigen-specific immunotherapy in patients with relapsing multiple sclerosis using different treatment protocols to induce tolerance to the antigen.

Walczak and co-workers (Walczak A, Siger M, Szczepanik M, Selmaj K. Transdermal application of myelin peptides in multiple sclerosis treatment. JAMA Neurol. 2013) have demonstrated efficacious antigen-specific therapy in multiple sclerosis using transdermal application of myelin peptides in human patients in a double-blind, placebo-controlled cohort study.

Lutterotti and co-workers have published a tolerization regimen in MS patients that uses a single infusion of autologous peripheral blood mononuclear cells chemically coupled with seven myelin peptides (Sci Transl Med. 2013 Jun. 5; 5(188):188ra75). The same team has also disclosed in a patent publication a similar regimen where the peptides were coupled to red blood cells (EP2205273).

Gholamzad and co-workers (Iranian Journal of Allergy, Asthma and Immunology 2017. 16(3):271-281) disclose a regimen comprising intravenous injection of Myelin Oligodendrocyte Glycoprotein (MOG)-coated PLGA microparticles having tolerogenic effects in Experimental Autoimmune Encephalomyelitis, a disease model for MS.

Pujol-Autonell and co-workers (Nanomedicine (Lond). 2017 June; 12(11):1231-1242. Doi: 10.2217/nnm-2016-0410) disclose a regimen with phosphatidylserine-liposome-based immunotherapy having therapeutic effect on multiple sclerosis disease model, with MOG-peptides as antigen.

Pearson and co-workers (Mol Ther. 2017 Jul. 5; 25(7):1655-1664. Doi: 10.1016/j.ymthe.2017.04.015) reported experimental protocols using antigenic MOG peptides conjugated to poly(lactide-co-glycolide) in relapsing-remitting experimental autoimmune encephalomyelitis (R-EAE), a murine model of multiple sclerosis. The polymer-conjugated peptides were effective in inhibiting disease.

Any of the regimens and protocols discussed above can be used with the composition of the first aspect, by modifying the antigenic peptide to be according to the first aspect.

Gene Therapy

Generally speaking, the nucleic acid of the second aspect is preferably included in a vector, operatively coupled to a promoter allowing expression in cells, preferably antigen-presenting cells, of the patient. The vector may be a gene transfer vector, or a viral vector known in the art, such as a retrovirus vector or, an adeno-associated virus vector or an adenovirus vector. A naked DNA gene transfer vector may be administered by any manner known in the art, including electroporation, gene gun, sonoporation, magnetofection or hydrodynamic delivery. Chemical methods for enhancing vector delivery that may be used include liposomes, lipoflexes, polymersomes, polyplexes, dendrimers, inorganic or organic nanoparticles or cell penetrating peptides.

Suitable promoters are known in the art, depending on the tissue where the expression of the sequence encoding for the therapeutic T-cell epitope is desired.

Keeler and coworkers (Mol Ther. 2018 Jan. 3; 26(1):173-183) have demonstrated that gene therapy-induced antigen-specific regulatory T-cells (Tregs) can inhibit neuro-inflammation and reverse disease in a MS, using a liver-targeting gene transfer vector that expresses full-length myelin oligodendrocyte glycoprotein (MOG) in hepatocytes. The materials and methods used may be applied to the method of treatment according to the second aspect, with the modification of the nucleic acid sequence expressed is replaced with a therapeutic T-cell epitope as defined herein for the second aspect.

Therapy Using Antigen-Presenting Cells

The antigen-presenting cells of the third aspect may be dendritic cells, monocytes, macrophages or B-cells, which may preferably be derived from peripheral blood mononuclear cells. Alternatively, the antigen-presenting cells may be microglia which may be CNS-derived. Preferably, the cells are autologous to the patient, but they may also be from a different individual which is donor-matched with respect to MHC receptors. Additionally, use of genetically engineered APC cell lines from a different individual or even different species is also envisioned, where the MHC receptors are engineered to match the patient.

The cells are exposed ex vivo to the composition of the third aspect (therapeutic T-cell epitopes) such that the cells take up the epitopes and present them to the patient's immune system after having been administered to the patient. Since the cells process and digest any polypeptides prior to displaying them om the surface, the epitope(s) may be given to the cells as part of a larger protein or several different proteins. It would also be equally applicable to transfect the cells with a nucleic acid construct encoding the therapeutic T-cell epitopes, such that the epitopes are expressed in the cells.

Phillips et al. (Front Immunol. 2017; 8: 1279) discuss in a review article planned and ongoing clinical studies using tolerogenic dendritic cells, for MS and other autoimmune diseases.

Jones and Hawiger (Front Immunol. 2017 May 9; 8:532. doi: 10.3389/fimmu.2017.00532) discuss experiments showing that neuroinflammation can be ameliorated or even completely prevented by the antigen-specific Treg cells formed extrathymically in the peripheral immune system (pTreg cells) during tolerogenic responses to relevant neuronal antigens, and the relevance of these findings for the treatment of MS.

Iberg et al. (Trends Immunol. 2017 November; 38(11):793-804. doi: 10.1016/j.it.2017.07.007) discuss how dendritic cell functions empowered by specific delivery of T cell antigens could be harnessed for tolerance induction in clinical settings.

Huang et al. report that autoantigen-pulsed dendritic cells induce tolerance to experimental allergic encephalomyelitis (EAE) in Lewis rats (Clin Exp Immunol (2000) 122(3):437-44.doi:10.104641365-2249. 2000.01398.x).

Menges et al. report that repetitive injections of dendritic cells matured with tumornecrosis factor alpha induce antigen-specific protection of mice from autoimmunity (J Exp Med (2002) 195(1):15-21. Doi:10.1084/jem.20011341).

The means and methods discussed in the above original studies and reviews (including the original studies cited therein) can be adapted to be used with compositions of the therapeutic T-cell-epitopes of the present invention.

Method for determining the degree of multiple sclerosis (MS) related autoimmunity

In a fourth aspect, the present invention provides a method for determining the degree of multiple sclerosis (MS) related autoimmunity in a test subject, comprising:
a. providing a test sample derived from the test subject comprising viable T-cells;
b. quantitating antigen-specific activation of the T-cells of the test sample in vitro in response to a test antigen comprising a specific T-cell epitope corresponding to a MS-antigen, wherein said T-cell epitope comprises an amino-acid sequence of n consecutive residues being:
   i. identical to a sub-sequence of SEQ ID NO: 5; or
   ii. differing from a sub-sequence of SEQ ID NO: 5 by no more than m residue substitutions, deletions and/or insertions;
   wherein n is at least 8, and m is 0, 1 or 2; and
c. comparing the quantitated antigen-specific activation to a relevant reference to determine the degree of MS-related autoimmunity in the test subject.

The method for determining the degree of multiple sclerosis (MS) related autoimmunity in a test subject of the fourth aspect may comprise:
a. providing a test sample derived from the test subject comprising viable T-cells;
b. quantitating antigen-specific activation of the T-cells of the test sample in vitro in response to a test antigen comprising a specific T-cell epitope corresponding to a MS-antigen; and
c. comparing the quantitated antigen-specific activation to a relevant reference to determine the degree of MS-related autoimmunity in the test subject;
wherein the MS-antigen is selected from the group consisting of FABP7 (SEQ ID NO: 1), PROK2 (SEQ ID NO: 2), RTN3 (SEQ ID NO: 3) and SNAP91 (SEQ ID NO: 4); wherein said T-cell epitope comprises an amino-acid sequence of n consecutive residues being:
   i. identical to a sub-sequence of the selected MS-antigen; or
   ii. differing from a sub-sequence of the selected MS-antigen by no more than m residue substitutions, deletions and/or insertions; and
wherein n is at least 8, and m is 0, 1 or 2.

It should be noted that the T-cell epitope can be included in practically any larger polypeptide test antigen (e.g. by way of genetic engineering) given that the antigen-presenting cells (APC) will digest the test antigen. Since the test antigen will be digested, the context in which the specific T-cell epitope is found in the test antigen makes no difference. Thus, any test antigen could be used, provided that the test antigen comprises a specific T-cell epitope corresponding to one of the novel MS-antigens FABP7, PROK2, RTN3 and SNAP91 as part of its sequence (all comprised in SEQ ID NO: 5).

As discussed under the background section, the shortest epitopes are typically 8 aa long but can be longer depending on the individual case. The present invention thus requires that the T-cell epitope has sequence identity or similarity to SEQ ID NO:5 (i.e. one of the novel MS-antigens) sharing similarity at least for a consecutive stretch of n amino acids.

The value of n may be at least 8, 9, 10, 11, 12, 13, 14, 15, 16, 17, 18, 19 or 20. Preferably, n is at least 11, more preferably n is at least 13, yet more preferably n is at least 15, still more preferably n is at least 17, even more preferably n is at least 19. Alternatively, n may be at least 50, preferably at least 75, or most preferably at least 100. n may be any interval formed from the aforementioned numbers, e.g. 8-22, 9-21, 8-17, 8-15, 8-12 or 8-100. Most preferably, n is 8-19, or 8-17 when m=0.

Preferably, the T-cell epitope is identical to the sub-sequence (m=0). However, it is in most cases possible to vary the sequence slightly and still have a functionally fully or substantially equivalent specific T-cell epitope corresponding to the MS-antigen. For instance, substituting an amino-acid for another with similar characteristics in terms of polarity, charge or hydrophobicity may be tolerable. Small insertions or deletions within the specific T-cell epitope may also be tolerable provided that they result in a functionally fully or substantially equivalent specific T-cell epitope corresponding to the selected MS-antigen.

Preferably, the differences only amount to substitutions, i.e. no deletions or insertions. More preferably, the differences only amount to substitutions involving substituting an amino-acid for another with similar characteristics in terms of polarity, charge and/or hydrophobicity. Preferably, m is 2, more preferably m is 1, most preferably m is 0.

Preferably, the antigen-specific activation is quantitated against at least two, three or four test antigens each comprising a specific T-cell epitope corresponding to a different MS-antigen selected from the group consisting of FABP7 (SEQ ID NO: 1), PROK2 (SEQ ID NO: 2), RTN3 (SEQ ID NO: 3) and SNAP91 (SEQ ID NO: 4). As shown in Example 2, not all MS-patients exhibit T-cell related autoimmunity to all of the novel MS-antigens. Thus, especially in a screening setting, it is advantageous to test for MS-related autoimmunity in a test subject against more than one specific T-cell epitope, derived from a different MS-antigen at the same time. It is also contemplated that determination of autoimmunity against the novel MS-markers can advantageously be combined with determination of other known MS-markers.

The test antigen comprising a specific T-cell epitope may be a peptide or peptidomimetic having at least 80%, preferably at least 90%, more preferably at least 95%, most preferably at least 98% sequence identity to any of SEQ ID NOs: 1-4, SEQ ID NO:5 or SEQ ID NO: 6.

Sub-Sequence in the Diagnostic Method

The sub-sequence of the fourth aspect may be included in i) residues 1-166 of SEQ ID NO: 5, ii) residues 167-295 of SEQ ID NO: 5, iii) residues 296-1327 of SEQ ID NO: 5, iv) residues 1328-2234 of SEQ ID NO: 5 or v) in residues 2235-2250 of SEQ ID NO: 5. Preferably, the sub-sequence is included in vi) residues 1-2234 of SEQ ID NO: 5.

The method of the fourth aspect may comprise quantitating antigen-specific activation in response to more than one different T-cell epitopes as defined above. Preferably, the more than one different T-cell epitopes are selected from two, three, four, five or six of the distinct intervals presented above as i)-vi).

It is preferable that method of the fourth aspect may comprise quantitating antigen-specific activation in response to more than one therapeutic T-cell epitopes, such as 2, 3, 4, 5, 6, 7, 8, 9, 10, 12, 14, 16, 18, 20, 30, 40, 50, 100 or more, each fulfilling the criteria set forth above.

The sub-sequence may be comprised in any one of the sequences of peptides in table 4. Preferably, the sub-sequence is comprised in any one of the sequences of peptides in table 4 where the HLA binding is indicated as “++” or “+++”, most preferably “+++”.

The sub-sequence may preferably be derived from a specific part of the MS-antigens described herein, corresponding to the specific antigen proteins used in the Examples.

Said sub-sequence may be included in the sequence of FABP7 (SEQ ID NO: 1) residues 1-82, residues 83-166, or residues 105-166.

Said sub-sequence may be included in the sequence of PROK2 (SEQ ID NO: 2) residues 34-74, or residues 106-128.

Said sub-sequence may be included in the sequence of RTN3 (SEQ ID NO: 3) residues 81-217, or residues 345-483.

Said sub-sequence may be included in the sequence of SNAP91 (SEQ ID NO: 4) residues 378-480, residues 481-572 or residues 584-691.

Test Subject

The subject may be a diagnosed MS-patient, or an individual suspected of having MS. Preferably, the subject is a human.

Test Sample

The test sample is preferably derived from a blood sample, and more preferably is a PBMC sample.

Most preferably, the test sample further comprises antigen-presenting cells (APC), that can be used to present the test antigen to the T-cells of the sample. Utilizing APCs from the same individual in the quantitation (more details below) eliminates any uncertainties arising from individual genetic variation in the MCH receptors on the APCs.

Details on Preferred Ways to Determine Specific T-Cell Activation

A T-cell reactivity platform useful in determining MS-related autoimmunity has previously been disclosed by the inventors in PCT/EP2016/081141.

The method of the present invention may further comprise:
a. Providing a viable antigen-presenting cell;
b. Contacting the test antigen with the antigen-presenting cell;
c. Contacting in vitro the test sample with the antigen-presenting cell contacted with the test antigen under conditions allowing antigen-specific activation of T-cells in response to an antigen presented by an antigen-presenting cell; and
d. Quantitating antigen-specific T-cell activation in the test sample.

It is preferred that the antigen-presenting cell is derived from the test subject.

The method may comprise providing the test antigen tightly associated to a phagocytable particle. The particle is phagocytosed by the antigen-presenting cell along with the test antigen. The test antigen is digested enzymatically by the APC and the digested antigen epitopes presented to the T-cells.

A particular advantage of having the test antigen tightly associated to a phagocytable particle is that any contaminating endotoxins can be removed by a denaturing wash. Unfortunately, a common problem with assays determining T-cell activation is that even low levels of endotoxins that come into contact with the T-cells result in an activation masking the normally very low level of antigen-specific activation. Only a small fraction of the T-cell population being tested reacts in an antigen-specific manner to a given antigen (in the order of 1/10000 in blood from a subject that has recently encountered the antigen), whereas a large fraction of the cells will respond to endotoxins creating a high level of background.

Given the ubiquitous endotoxin contamination this can be a substantial issue in practical terms.

Thus, the method may further comprise the steps (a′) tightly associating the test antigen to a phagocytable particle and/or (a″) subjecting the test antigen associated with a particle to a denaturing wash resulting in an endotoxin level low enough to not interfere with the subsequent steps.

The particle preferably has a largest dimension of less than 5.6 μm, preferably less than 4 μm, more preferably less than 3 μm, even more preferably in the interval 0.5-2 μm or most preferably about 1 μm. The particle is preferably substantially spherical.

The denaturing wash may involve subjecting the particle with the associated test antigen to a high pH, such as at least pH 13, more preferably at least pH 14, most preferably at least pH 14.3. The denaturing wash may involve subjecting the particle with the associated test antigen to a low pH. The denaturing wash may involve subjecting the particle with the associated test antigen to a high temperature, such as at least 90° C., more preferably at least 92° C., most preferably at least 95° C. The denaturing wash may involve subjecting the particle with the associated test antigen to a denaturing agent, such as urea or guanidine hydrochloride at a sufficient concentration, such as at least 5M, 6M, 7M or 8M.

Preferably, the denaturing wash results in an endotoxin amount in the test antigen being such that in the method, the final concentration of endotoxin is less than 100 pg/ml, preferably less than 50 pg/ml, more preferably less than 25 pg/ml and most preferably less than 10 pg/ml.

It is advantageous if the particle has paramagnetic properties, allowing easy handling by magnetic retention.

Preferably, the test antigen is covalently linked to the particle or linked to the particle via a metal chelate.

Antigen-Presenting Cell (APC) and the T-Cell Sample

In the context of the present invention, the APC is a professional antigen presenting cell, such as a monocyte/macrophage or a dendritic cell. The APC may be a primary cell or an immortalized cell.

The APC must be compatible with the T-cells of the T-cell sample, such that they are capable of presenting antigens to the T-cells in an antigen-specific context (MHC restricted) that the T-cells can react to. The APC and the T-cell sample are preferably obtained from the same species and donor-matched with respect to MHC receptors. However, use of genetically engineered APCs from a different species is also envisioned.

If the antigen-presenting cell and the T-cell sample are derived from the same individual, any potential for a mismatch between the APC and the T-cells is avoided.

The antigen-presenting cell and the T-cell sample may be derived from the same blood sample, which is advantageous from a practical point of view. The antigen-presenting cell and the T-cell sample may be derived from a PBMC-sample from the same individual. Obtaining PBMC from peripheral blood samples is a routine protocol, which provides a handy source for both APCs and T-cells at the same time and from the same individual.

The PBMC sample may be freshly used or subjected to freezing. The possibility of using frozen cells is of great practical advantage from a logistical point of view.

The T-cell sample may be derived from a tumour, preferably a lymphatic vessel in a tumour.

The T-cell sample may also be derived from ascites.

The T-cell sample may comprise whole PBMCs including both CD4+ and CD8+ T-cells, purified T-cell populations, or PBMCs depleted of (a) particular T-cell population(s).

Quantitating Antigen-Specific T-Cell Activation

The quantitation of antigen-specific T-cell activation may comprise the steps of:
a. Providing a phagocytable particle, having the test antigen tightly associated thereto, wherein the particle with the associated test antigen has been subjected to a denaturing wash resulting in an endotoxin level low enough to not interfere with the subsequent steps;
b. Providing a viable antigen-presenting cell;
c. Contacting the washed particle with the antigen-presenting cell under conditions allowing phagocytosis of the particle by the antigen-presenting cell;
d. Providing the test sample to be assayed comprising viable T-cells;
e. Contacting in vitro the test sample with the antigen-presenting cell contacted with the particle under conditions allowing antigen-specific activation of T-cells in response to an antigen presented by an antigen-presenting cell; and
f. Quantitating antigen-specific T-cell activation in the test sample.

Quantitating the antigen-specific T-cell activation in the test sample may performed using an ELISpot or a FluoroSpot-technique, or by measuring T-cell proliferation. It is important to note that while the present Examples use expression of particular cytokines as a readout, there are numerous additional ways of determining antigen-specific T-cell activation that could be used. For instance, using T-cell proliferation as a readout (utility is demonstrated earlier in PCT/EP2016/081141) can be used to eliminate need to measure any specific cytokine.

Quantitating the antigen-specific T-cell activation in the test sample may preferably comprise determining the T-cell response by measuring secretion of IFN-γ, IL-17 and/or IL-22. Among these, IL-17 and IL-22 are particularly preferred as they give the most robust results (see Table 3)

Preferably, quantitating T-cell activation in the test cell sample involves determining the fraction of activated T-cells in the sample. Quantitating T-cell activation in the test cell sample may involve determining the ratio of activated T-cells in the sample detected using two different measures. The number may be normalized by numerical operations, such as taking a logarithm or square root. One particularly preferred quantitation involves determining the measure obtained by dividing the number of IL-17 positive cells from the square root of the number of IFN-γ positive cells.

Relevant Reference

Preferably, the relevant reference to which the quantitated antigen-specific activation is compared to is comparably quantitated antigen-specific activation in a reference sample from a reference subject free of pathological MS-related autoimmunity.

The reference may be a mean value of comparably quantitated antigen-specific activation in a set of reference samples from a set of reference subjects free of pathological MS-related autoimmunity. Said set may comprise at least 10 reference subjects.

The reference may be comparably quantitated antigen-specific activation in a sample from the same subject taken at a different point in time.

Diagnostic Applications

The diagnostic utility of the method was shown in Examples 3-7. As demonstrated by the receiver-operator-characteristic (ROC)-analysis there is always a trade-off between sensitivity and selectivity. If the threshold for concluding the presence of a pathology is set lower, the sensitivity is increased (i.e. the number of false negatives is reduced), but at the cost of lowering selectivity (i.e. the number of false positives is increased). If the threshold is raised instead, the sensitivity is decreased and selectivity increased. Depending on the setting, the tolerances for false negatives and false positives will differ. For instance, in a population-wide screening where millions of people are tested, the number of false positives must be very low, otherwise the number of patients needing a follow-up will be overwhelming. On the other hand, if a diagnostic test is used as part of a specialist evaluation of a single patient, where several other factors will be weighed in before reaching a diagnosis, a much larger proportion of false positives may be regarded tolerable. Thus, the threshold for making a conclusion will is most cases need to be set considering the setting in which the analysis is made, and determining a generally applicable threshold is neither appropriate nor necessary.

An increased degree of MS-related autoimmunity may be concluded if the quantitated antigen-specific activation in the test sample is at least 2 times, preferably 3 times, more preferably 5 times, most preferably 10 times higher compared to the reference.

An increased degree of MS-related autoimmunity may be concluded if the quantitated antigen-specific activation in the test sample is higher than the mean of a set of comparably quantitated reference samples from a set of reference subjects free of pathological MS-related autoimmunity by 2 times the standard deviation of the set of reference samples.

An increased degree of MS-related autoimmunity may be concluded if the quantitated antigen-specific activation in the test sample is statistically significantly higher than the reference with a p value of less than 0.05 calculated with Student's T-test.

An increased degree of MS-related autoimmunity may be concluded if the quantitated antigen-specific activation in the test sample is statistically significantly higher than the reference with a p value of less than 0.05 calculated with Mann-Whitney U-test.

Applications in Specific Settings

There is provided a method according to the fourth aspect, wherein:
a. the method is for diagnosing MS in the subject;
b. the reference is representative of comparably quantitated antigen-specific activation in reference subjects free of pathological multiple sclerosis related autoimmunity; and
c. a higher degree of quantitated antigen-specific activation in the test sample compared to the reference is indicative of multiple sclerosis in the test subject.

Said method is specifically adapted for detecting the presence of MS in a patient.

There is provided a method according to the fourth aspect, wherein:
a. the method is for following course of MS in the subject;
b. the reference is representative of comparably quantitated antigen-specific activation in a sample taken at a different point in time (preferably earlier) from the same subject; and
c. a higher degree of quantitated antigen-specific activation in the test sample compared to the reference is indicative of higher multiple sclerosis disease activity in the test subject, and vice versa.

Said method is specifically adapted for following the course of MS in a subject already known to suffer of MS.

There is provided a method according to the fourth aspect, wherein:
a. the method is for making prognosis of the course of MS in the subject;
b. the reference is representative of comparably quantitated antigen-specific activation in a sample taken at an earlier point in time from the same subject; and
c. a higher degree of quantitated antigen-specific activation in the test sample compared to the reference is indicative of increasing multiple sclerosis disease activity in the test subject, and vice versa.

Said method is specifically adapted for making prognosis of MS course in a subject already known to suffer from MS. Given that MS is characterized by relapses with intermittent recovery, it is of value to detect an oncoming relapse in advance, to allow a treatment to be administered in advance.

There is provided a method according to the fourth aspect, wherein:
a. the method is for evaluating the response of the subject to a therapeutic treatment;
b. the reference is representative of comparably quantitated antigen-specific activation in a sample taken from the same subject prior to administration of the therapeutic treatment to be evaluated;
c. the test sample is from the same subject after initiation of the therapeutic treatment; and
d. a lower degree of quantitated antigen-specific activation in the test sample compared to the reference is indicative of therapeutic efficacy against multiple sclerosis disease activity in the test subject, and unchanged or higher degree of quantitated antigen-specific activation is indicative of lack of therapeutic efficacy against multiple sclerosis disease activity in the test subject.

Said method is specifically adapted for evaluating response of the subject to a therapeutic treatment. In particular, the method is valuable in conducting clinical trials, for choosing the right drug for an individual patient and for dose-finding for an individual patient.

Use of a Peptide or Peptidomimetic

In a fifth aspect, there is provided an use of a peptide or peptidomimetic in the diagnosis or treatment of MS, wherein the peptide or peptidomimetic comprises a specific T-cell epitope comprised in SEQ ID NO: 5, wherein said T-cell epitope comprises an amino-acid sequence of n consecutive residues differing from a sub-sequence of the selected MS-antigen by no more than 0, 1 or 2 residue substitutions, deletions and/or insertions. The use may be in vitro, in particular for the diagnostic use.

The sub-sequence may be selected from residues 1-166 of SEQ ID NO:5, residues 167-295 of SEQ ID NO:5, residues 296-1327 of SEQ ID NO:5, residues 1328-2234 of SEQ ID NO:5, residues 2235-2250 of SEQ ID NO: 5 or residues 1-2234 of SEQ ID NO: 5.

Preferably, the T-cell epitope may correspond to a MS-antigen selected from the group consisting of FABP7 isoform 2 (SEQ ID NO: 1), PROK2 (SEQ ID NO: 2), RTN3 (SEQ ID NO: 3), SNAP91 (SEQ ID NO: 4) and FABP7 isoform 1 (SEQ ID NO: 6).

The value of n may be 8, 9, 10, 11, 12, 13, 14, 15, 16, 17, 18, 19 or 20. Preferably, n is at least 11, more preferably n is at least 13, yet more preferably n is at least 15, still more preferably n is at least 17, even more preferably n is at least 19. Preferably m is 1, more preferably m is 0.

The T-cell epitope of the fifth aspect may be as defined for therapeutic T-cell epitope of the first aspect.

General Aspects Relating to the Present Disclosure

The term “comprising” is to be interpreted as including, but not being limited to. The arrangement of the present disclosure into sections with headings and subheadings is merely to improve legibility and is not to be interpreted limiting in any way, in particular, the division does not in any way preclude or limit combining features under different headings and subheadings with each other.

All references are hereby incorporated by reference.

### EXAMPLES

The following examples are not to be regarded as limiting. For further information on the experimental details, the skilled reader is directed to a separate section titled Materials and Methods.

### Example 1: Identification of Autoantigens in Multiple Sclerosis by FluoroSpot Autoantigen Screening

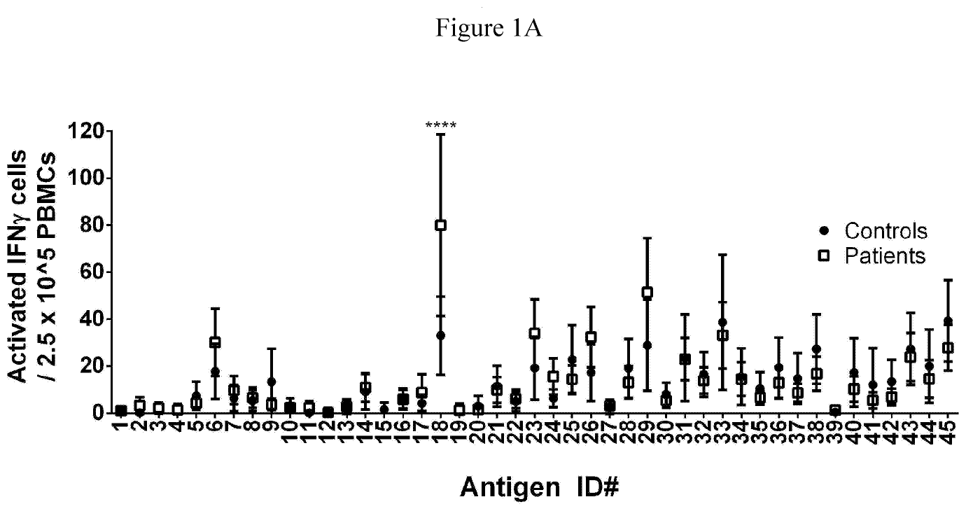

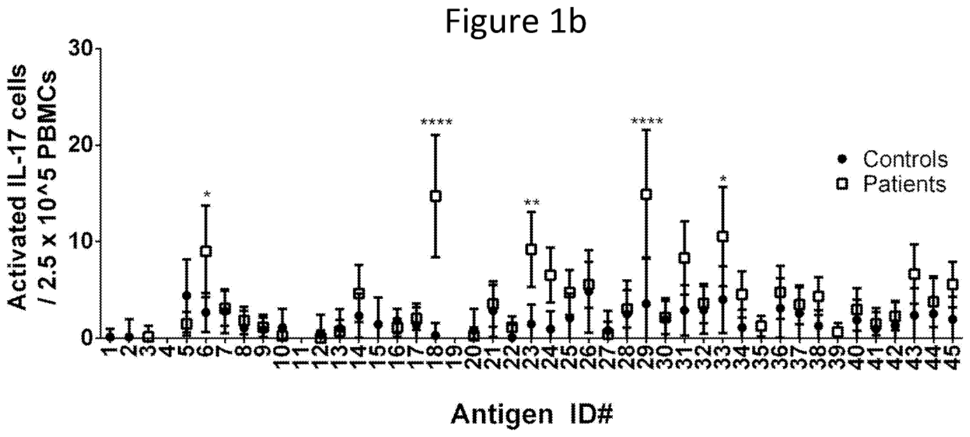

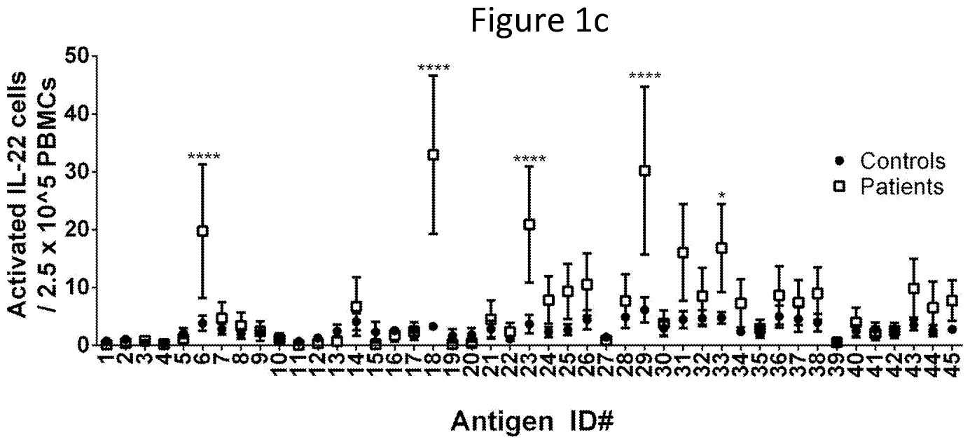

The inventors measured T-cell activation in response to a library of 125 PrESTs divided into 45 pools and coupled to beads, using IFNγ/IL-22/IL-17A FluoroSpot as assay for T-cell activation. The screening identified possible autoantigens by detecting those antigen pools that stimulate a higher T-cell response in PBMCs from MS patients compared to healthy controls. As seen in FIG. 1, in this screening of 16 patients and 9 healthy controls, statistically significant difference between mean patient and mean control number of activated cells could be seen when analysed using a Two-Way ANOVA. For IL-22 secreting activated T-cells difference was seen for 5 antigen pools, antigen #6 (P<0.0001), antigen #18 (P<0.0001), antigen #23 (P<0.0001), antigen #29 (P<0.0001) and antigen #33 (P<0.05) (panel a). For IL-17 secreting T-cells a difference between patients and controls could be seen for the same 5 antigens, antigen #6 (P<0.05), antigen #18 (P<0.0001), antigen #23 (P<0.01), antigen #29 (P<0.0001) and antigen #33 (P<0.05) (panel b). For IFNγ a difference could be seen only for antigen #18 (p<0.0001) (panel c).

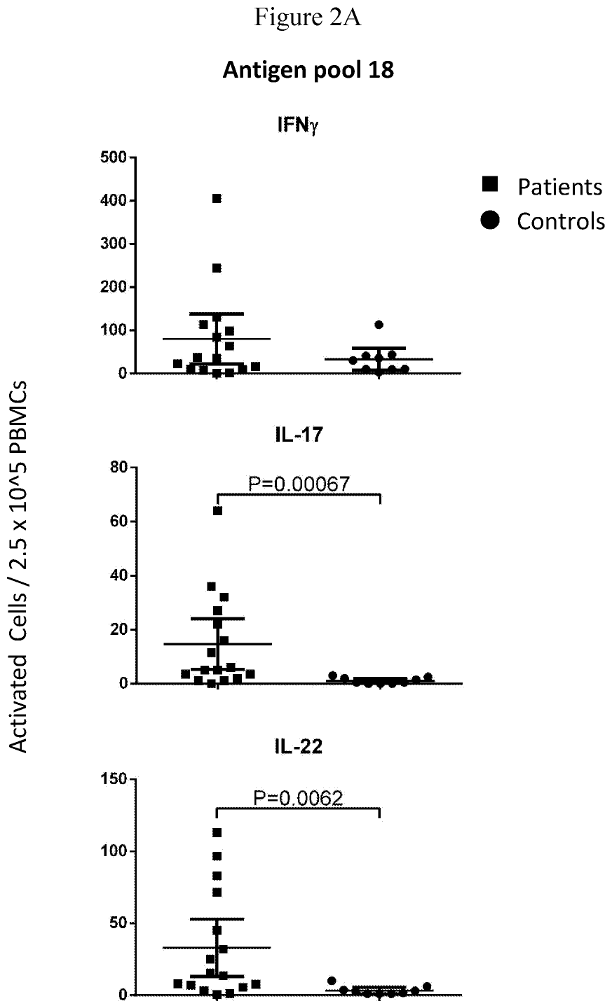

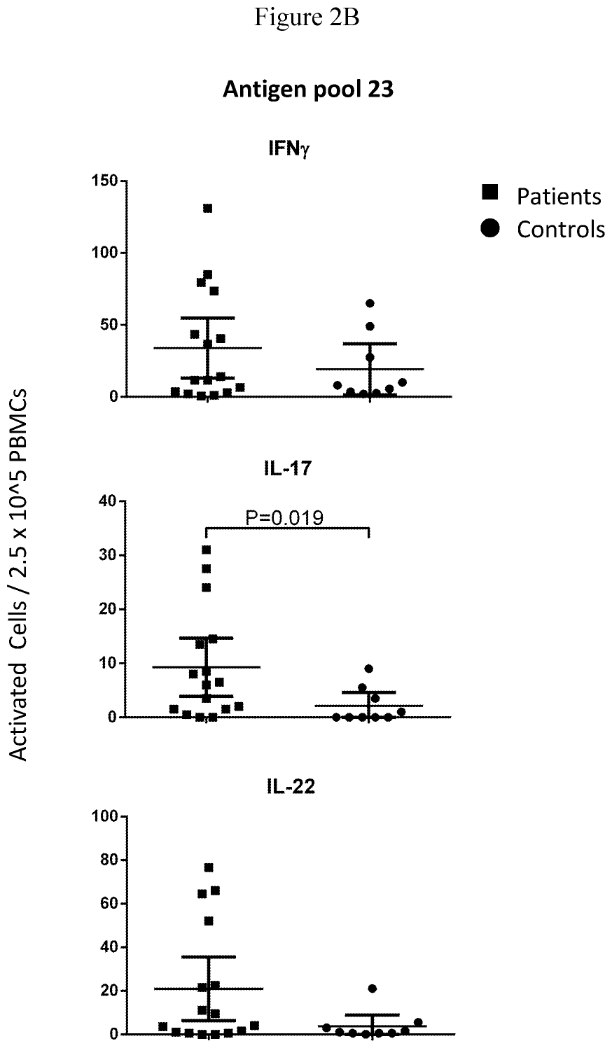

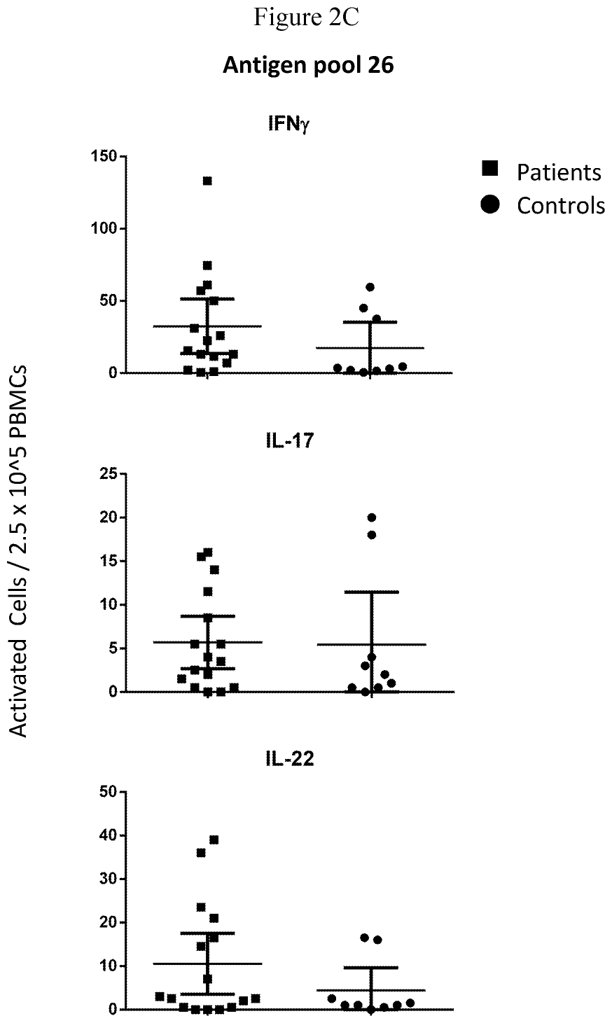

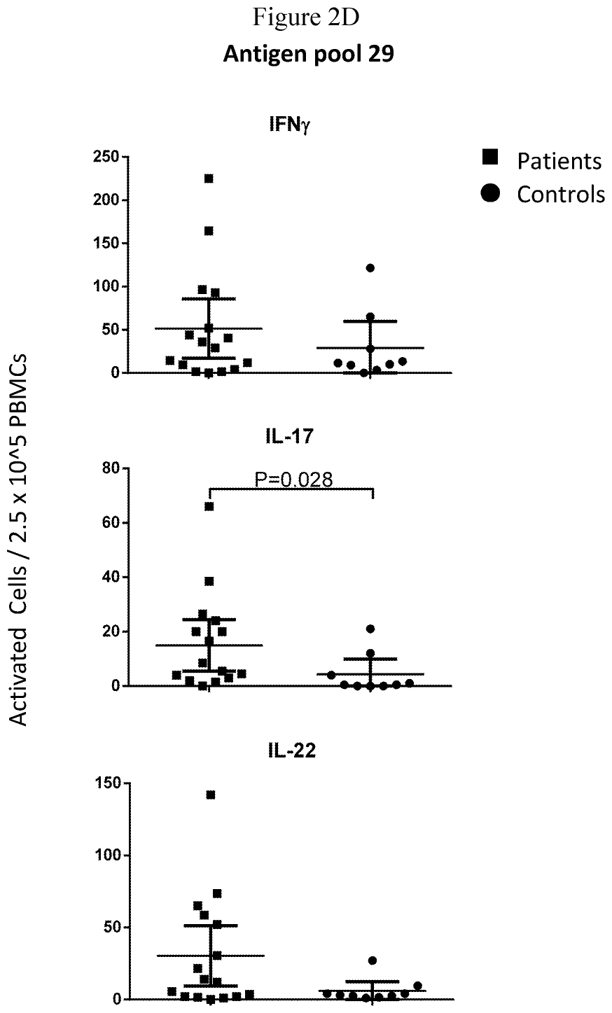

FIG. 2 shows the same data presented as the individual patient and control activation towards the above antigen pools.

### Example 2: Determining the Immunogenic Antigen Contained in the Antigen Pools

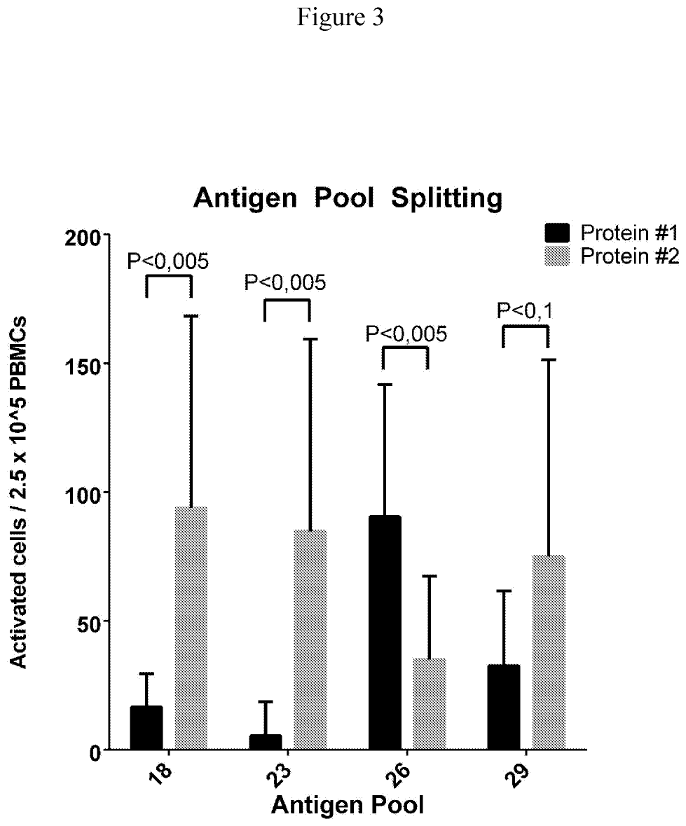

Antigen pools 18, 23, 26 and 29 described in example 1 were split up into the individual proteins included therein. PBMCs from 4 patients which showed a high degree of T-cell activation towards these specific antigen pools in example 1 were again tested for activation against the individual proteins. The method and protocol used was the same as in Example 1. In each pool, one specific protein elicited the clear majority of the T-cell activation. The proteins tested were: antigen pool 18, #1 CYB561D2 and #2 FABP7 (SEQ ID NO: 1). Antigen pool 23, containing #1 NOVA2 and #2 PROK2 (SEQ ID NO: 2). Antigen pool 26, containing #1 RTN3 (SEQ ID NO: 3) and #2 SDK2. Antigen pool 29, containing #1 SNAP25 and #2 SNAP91 (SEQ ID NO: 4). Results presented in FIG. 3.

### Example 3: Grouping Patients Based on Reactivity Profiles

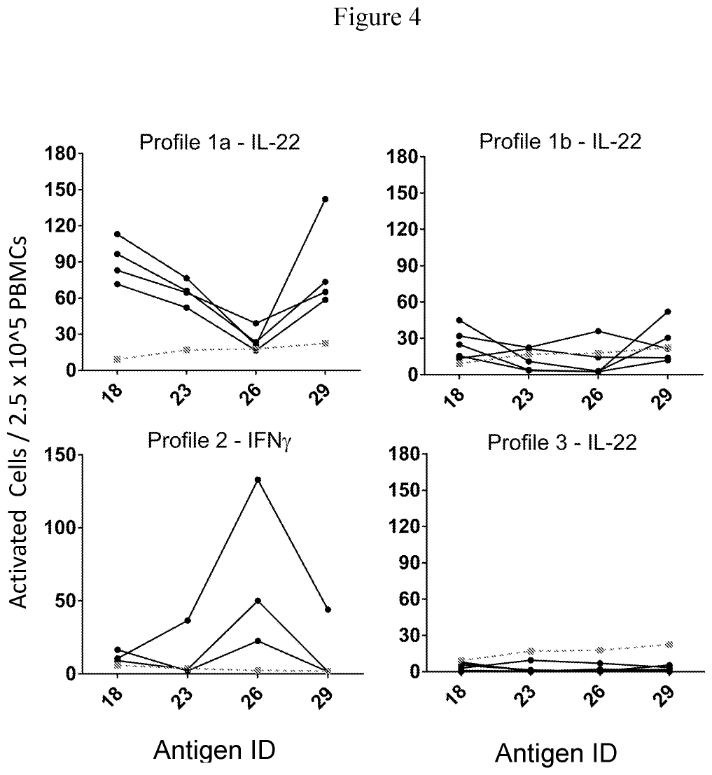

When analyzing the T-cell activation against the 4 candidate autoantigens at the level of an individual subject, four distinct reactivity profiles were observed among patients. Four patients responded with significant activation of cells producing all cytokines when stimulated with all candidate antigens except #26, named “profile 1a”. Five patients responded with significant activation of cells producing all cytokines against #18 and 1 or 2 more antigens, named “profile 1b”. Three patients responded with exclusively activated IFNγ-producing cells against #26, named “profile 2”. Four patients were non-responders, named “profile 3”. The profiles are presented in FIG. 4.

The patients in profile 2 were too few to generate a clear statistical difference between the mean patient and control activation (see FIGS. 1 and 2C) but had a remarkably unique profile not found in any of the healthy controls.

### Example 4: Demonstration of Diagnostic Use

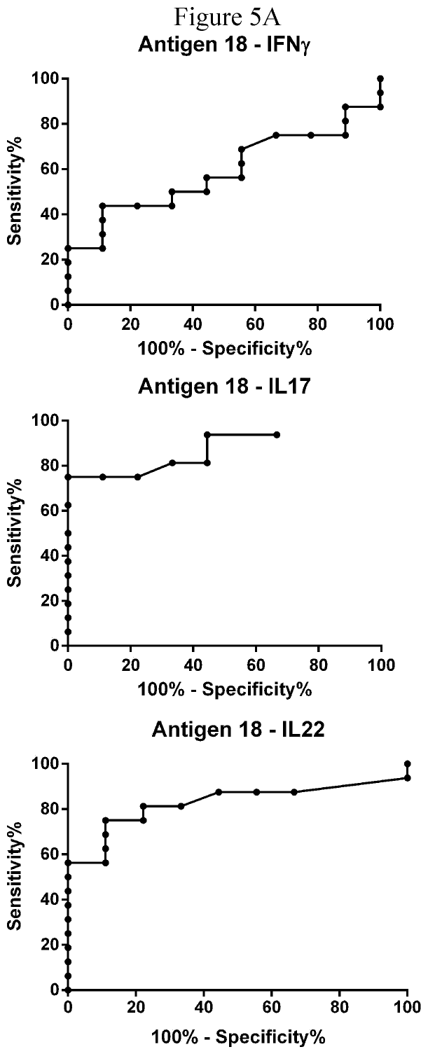

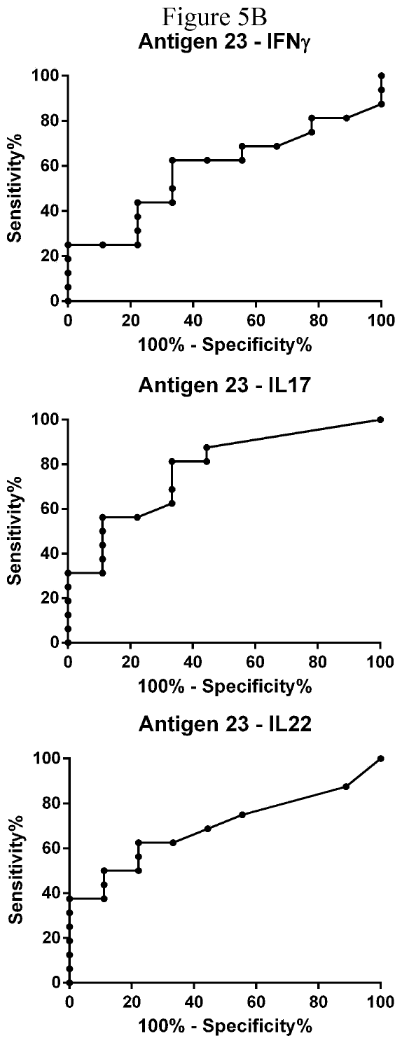

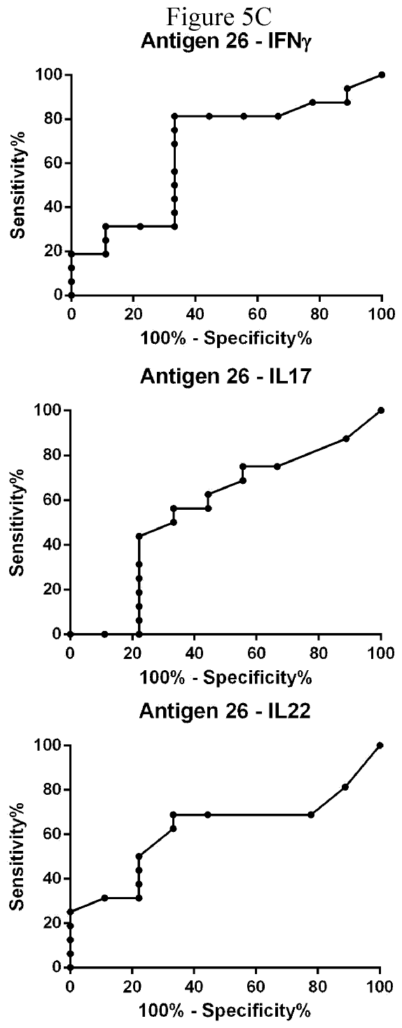

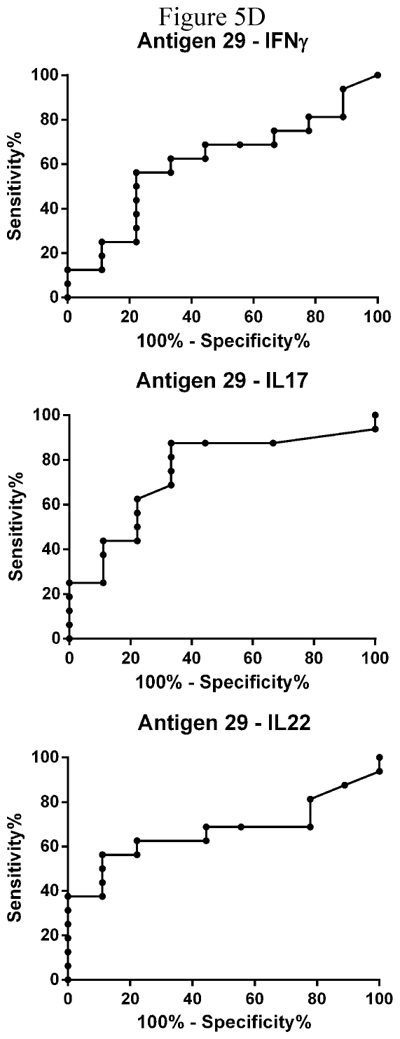

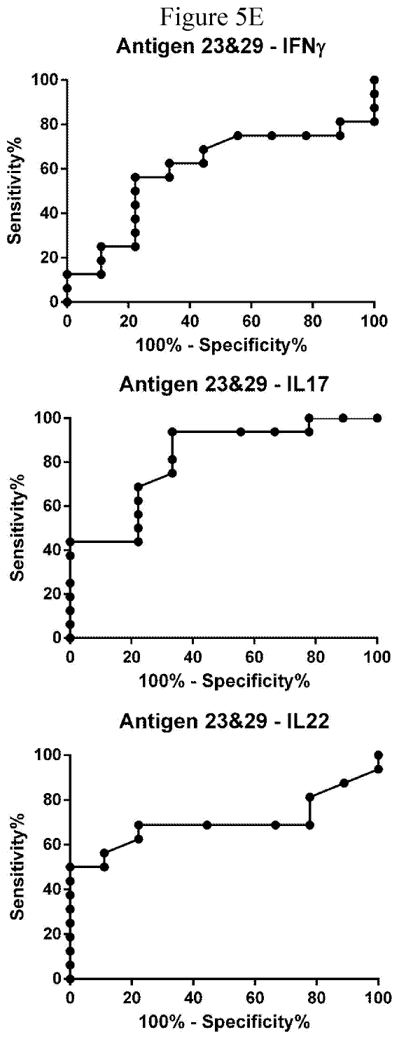

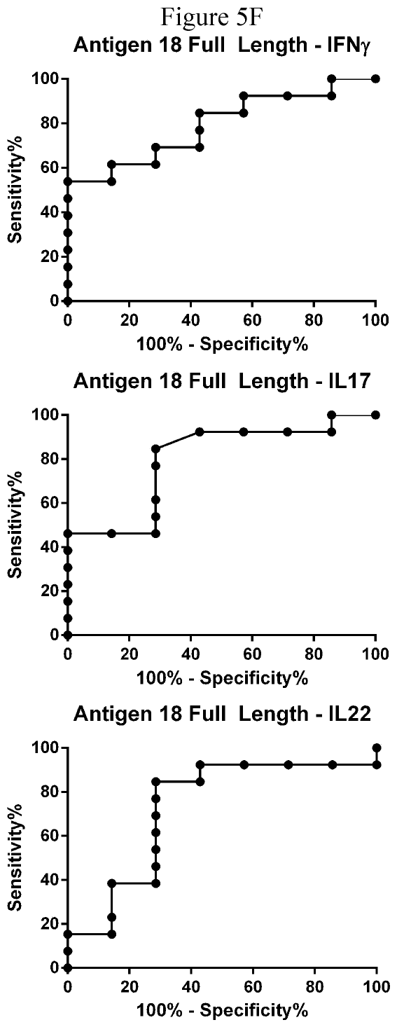

T-cell activation towards these antigens can used as a diagnostics tool and biomarker for multiple sclerosis. Based on the data from Example 1, receiver-operator characteristic (ROC)-curves were created to estimate sensitivity and specificity if T-cell activation were to be used as a diagnostics tool. These graphs are presented in FIG. 5A-F and the data in Table 1 and 3. Activation of IL22 and IL17 producing T-cells when stimulated with antigen #18 (containing FABP7 PrESTs) or the full length FABP7 isoform 2 (SEQ ID NO: 1) is particularly promising, reaching a sensitivity and specificity of about 80% respectively. Testing patient and control PBMCs to a mix of all of these antigens would very likely increase these numbers further. As can be seen in table 2, a combination of antigen 23 and 29 gives a more sensitive test compared to each antigen on its own.

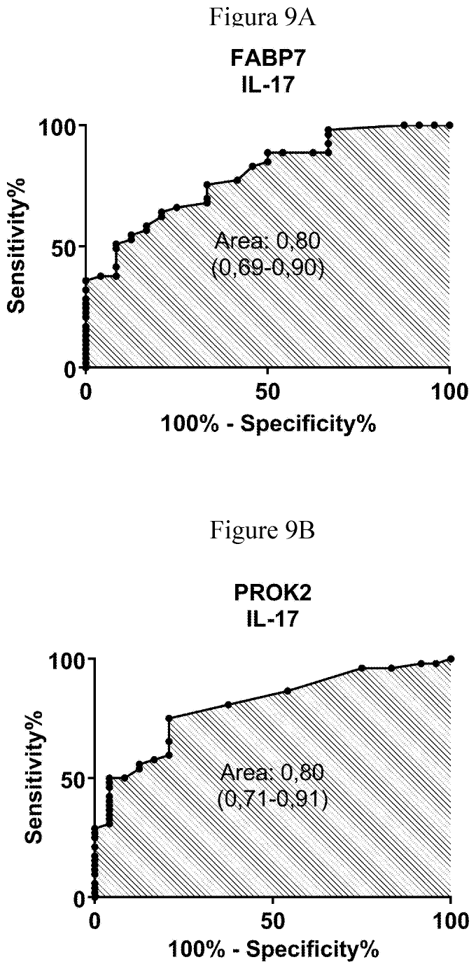

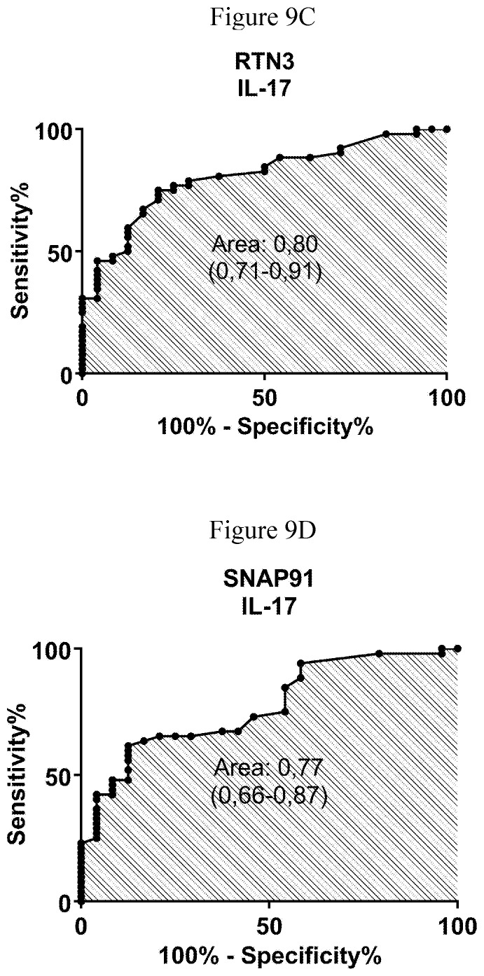

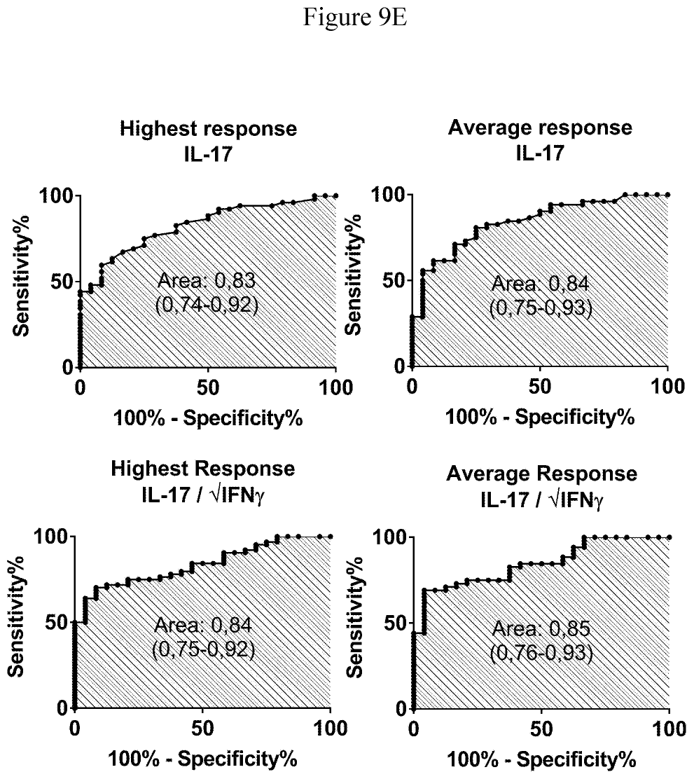

The full-length proteins can also be used in a similar way, see FIG. 9A-D. Utilizing all four full-length antigens, using each individual's average response to the four, increases the sensitivity and specificity further (FIG. 9E). Combining different readouts, such as analyzing the ratio between IL-17 and IFNγ, can increase the sensitivity of the test further (FIG. 9E, Table 2).

TABLE 1
Sensitivity and specificity when using a specific number of
activated IL17 producing cells as cut-off. For this
particular antigen and readout the sensitivity and
specificity reaches 75% and 100% when setting the
cut-off for a “positive test” at >3 IL-17 spots.

| Number of IL-17 activated | Sensitivity | Specificity |
|---|---|---|
| cells for 18 PrEST | (%) | (%) |
| 0.25 | 93.75 | 33.33 |
| 0.75 | 93.75 | 55.56 |
| 1.25 | 81.25 | 55.56 |
| 1.75 | 81.25 | 66.67 |
| 2.25 | 75 | 77.78 |
| 2.75 | 75 | 88.89 |
| 3.25 | 75 | 100 |
| 4.25 | 62.25 | 100 |
| 5.5 | 50 | 100 |

TABLE 2
Sensitivity and specificity when using the ratio between
average number of IL-17 producing cells and average
number of IFNγ producing cells reactive towards the antigens.
ROC-curve based on the ratio of number of cells producing
IL-17 and IFNγ rather than absolute number generates an even
stronger diagnostic test. The ratio is calculated as average
number of IL-17 producing cells divided by the square root of the
average number of IFNγ producing cells reactive to the antigens.

| Ratio: IL-17/square | Sensitivity | Specificity |
|---|---|---|
| root of IFNγ cells | (%) | (%) |
| Ratio: >0.24 | 100 | 33 |
| Ratio: >0.44 | 94 | 37 |
| Ratio: >0.78 | 88 | 42 |
| Ratio: >1.02 | 85 | 58 |
| Ratio: >1.15 | 83 | 63 |
| Ratio: >1.67 | 75 | 79 |
| Ratio: >1.82 | 73 | 83 |
| Ratio: >2.14 | 69 | 95 |
| Ratio: >3.45 | 44 | 100 |

TABLE 3
Descriptive parameter for the ROC-curves presented in FIG. 5A-E.

| Antigen | Cytokine | Area | Std. Error | Cl 95% | P-value |
|---|---|---|---|---|---|
| 18 PrESTs | IFNγ | 0.59 | 0.11 | 0.36-0.81 | 0.48 |
| | Il 17 | 0.88 | 0.07 | 0.74-1.0 | 0.0022 |
| | IL 22 | 0.83 | 0.085 | 0.66-0.99 | 0.0078 |
| 23 PrESTs | IFNγ | 0.58 | 0.12 | 0.36-0.81 | 0.50 |
| | Il 17 | 0.77 | 0.097 | 0.58-0.97 | 0.025 |
| | IL 22 | 0.69 | 0.10 | 0.49-0.90 | 0.11 |
| 26 PrESTs | IFNγ | 0.66 | 0.12 | 0.43-0.89 | 0.19 |
| | Il 17 | 0.56 | 0.13 | 0.30-0.81 | 0.65 |
| | IL 22 | 0.62 | 0.11 | 0.38-0.84 | 0.34 |
| 29 PrESTs | IFNγ | 0.61 | 0.12 | 0.38-0.84 | 0.38 |
| | Il 17 | 0.74 | 0.11 | 0.54-0.95 | 0.048 |
| | IL 22 | 0.67 | 0.11 | 0.45-0.88 | 0.17 |
| 23 + 29 PrESTs | IFNγ | 0.59 | 0.12 | 0.36-0.83 | 0.44 |
| | Il 17 | 0.82 | 0.09 | 0.64-0.99 | 0.01 |
| | IL 22 | 0.70 | 0.11 | 0.49-0.91 | 0.11 |
| FABP7 isoform 2. | IFNγ | 0.79 | 0.10 | 0.59-0.99 | 0.036 |
| Full length | Il 17 | 0.80 | 0.11 | 0.59-1.0 | 0.032 |
| | IL 22 | 0.73 | 0.13 | 0.47-0.99 | 0.10 |

### Example 5: Validation of FABP7 as MS-Antigen Using Full Length Recombinant Version

A recombinant form of human FABP7 isoform 2 (SEQ ID NO: 1) was produced in E. coli. The protein was produced in house and according to standard E.coli recombinant protein protocols. It was purified via his-tag purification and coupled to paramagnetic beads and washed according to previously described protocols. 13 patients (of which 7 consisted of new samplings of previous included patients and 6 not previously tested) and 7 controls (none included in previous tests) were tested for T-cell activation in the FluoroSpot assay previously described.

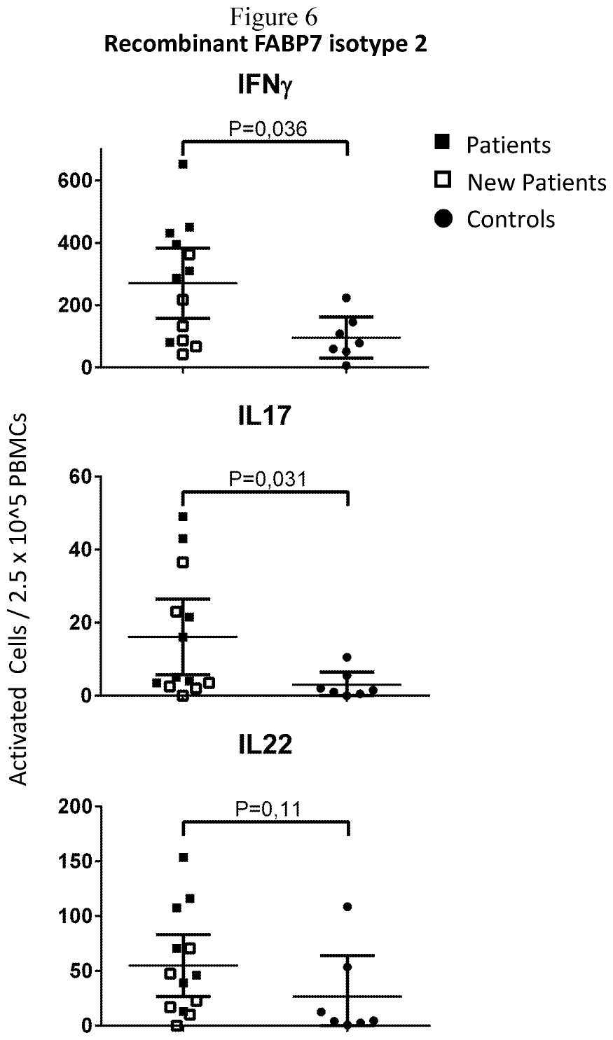

Results are presented in FIG. 6. The results from the FABP7 PrESTs in examples 1 and 3, and full length recombinant version used in this example closely resemble each other.

### Example 6: Identification of the Specific T-Cell Epitope

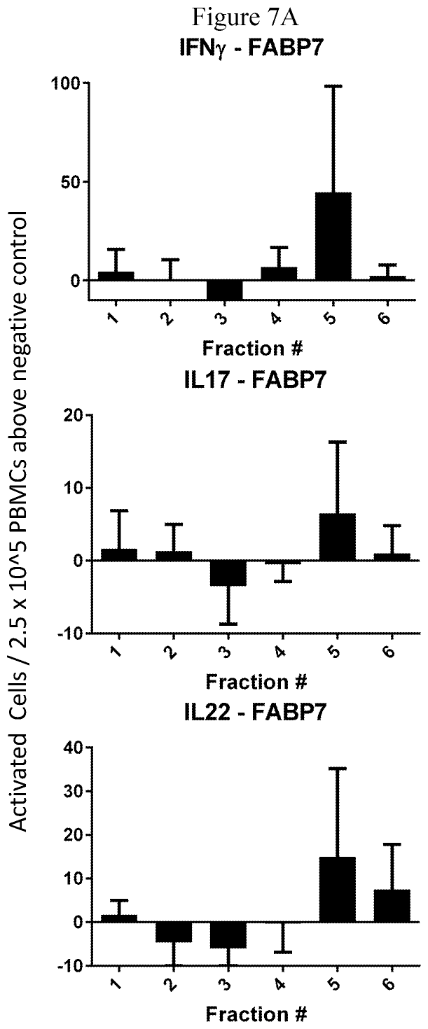

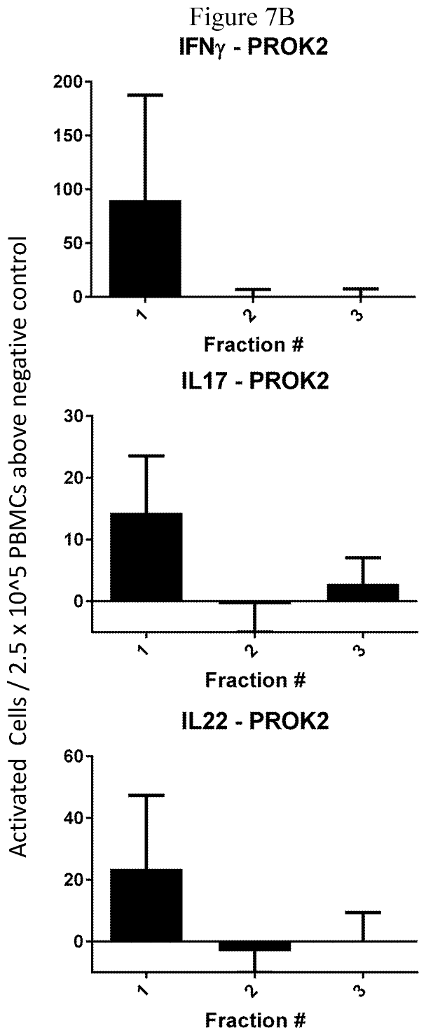

Overlapping peptides (15 aa long, with 10 aa overlap) covering the whole span of FABP7 the largely overlapping isoforms 2 and 1 (SEQ ID NO: 1 and SEQ ID NO:6, respectively) and PROK2 (SEQ ID NO: 2) were pooled into 6 fractions for FABP7 and 3 fractions for PROK2, and used to stimulate PBMCs in a FluoroSpot-assay. PBMCs from 6 patients were tested against these pools. As FIGS. 7A and 7B shows, the patients reacted higher to a single pool for each antigen (pool 5 for FABP7, pool 1 for PROK2). Compared to the previous examples which used longer proteins, this example shows that short peptides, with amino acid sequences containing specific T-cell epitopes, can also be used to identify autoreactive T-cells in MS-patients.

Splitting the positive peptide pools further and iteratively repeating the experiment will allow even more detailed identification of the specific T-cell epitope within each MS-antigen. Peptides even shorter than 15 aa can then be designed and tested to fully elucidate the specific T-cell epitope.

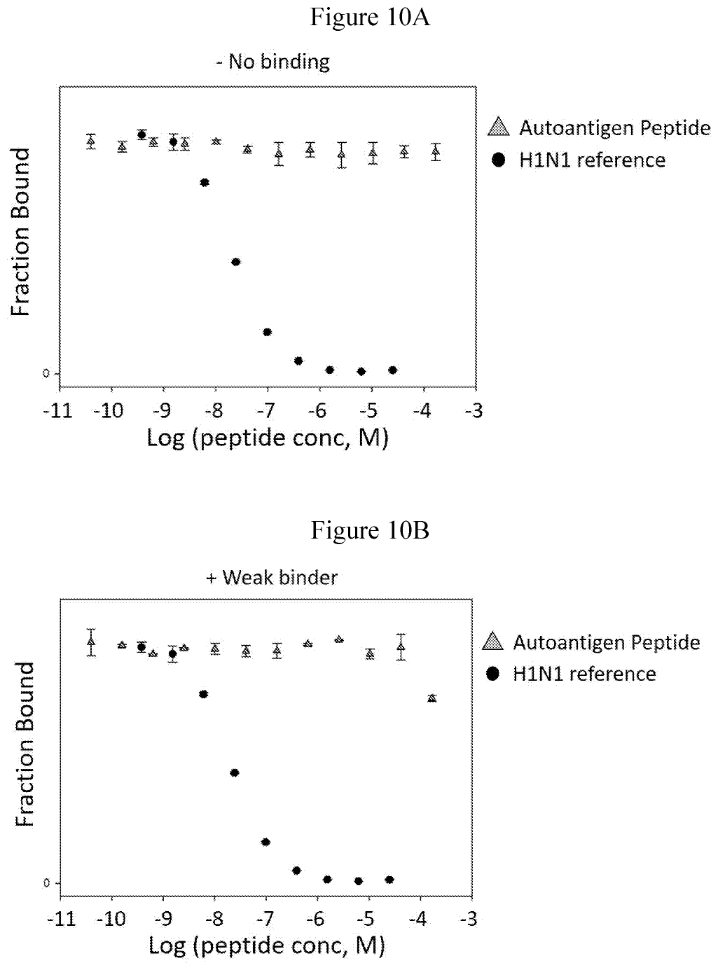

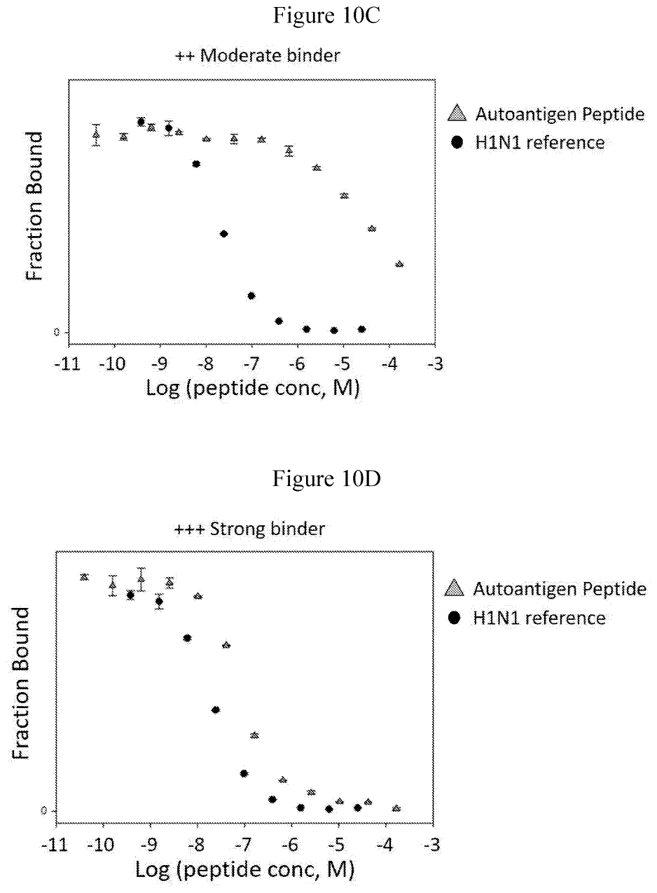

Another, complementary way of discovering possible T-cell epitopes is by conducting HLA-binding experiments. The same overlapping peptides were tested for their binding-affinity to the multiple sclerosis associated HLA DRB5 01:01 molecule in a competition binding assay. The binding-affinity was then compared to that of a known strong binder peptide derived from the H1N1 influenza virus. For both FABP7 and PROK2, the strongest binding peptide was found in the same peptide-pool that the patients reacted too, indicating that it is a T-cell epitope candidate. Results are presented in table 4. Representative examples of the different binding affinities are shown in FIG. 10A-E.

TABLE 4
Affinity of overlapping peptides from FABP7
and PROK 2 to HLA DRB5 01:01.
Affinity was tested in a competitive binding assay.
Legend: − No binding, + weak binding, ++ moderate binding,
+++ strong binding.

| Pool from | Peptide | | Binding |
|---|---|---|---|
| Fluorospot | # | Amino acid sequence | affinity |
| Pool 1 | #1 | Aa 02-16—SEQ ID NO: 1 | + |
| FABP7 | #2 | Aa 07-21—SEQ ID NO: 1 | + |
| | #3 | Aa 12-26—SEQ ID NO: 1 | − |
| | #4 | Aa 17-31—SEQ ID NO: 1 | ++ |
| | #5 | Aa 22-36—SEQ ID NO: 1 | − |
| | #6 | Aa 27-41—SEQ ID NO: 1 | + |
| Pool 2 | #7 | Aa 32-46—SEQ ID NO: 1 | − |
| FABP7 | #8 | Aa 37-51—SEQ ID NO: 1 | − |
| | #9 | Aa 42-56—SEQ ID NO: 1 | − |
| | #10 | Aa 47-61—SEQ ID NO: 1 | + |
| | #11 | Aa 52-66—SEQ ID NO: 1 | − |
| | #12 | Aa 57-71—SEQ ID NO: 1 | − |
| Pool 3 | #13 | Aa 62-76—SEQ ID NO: 1 | − |
| FABP7 | #14 | Aa 67-81—SEQ ID NO: 1 | − |
| | #15 | Aa 72-86—SEQ ID NO: 1 | − |
| | #16 | Aa 77-91—SEQ ID NO: 1 | − |
| | #17 | Aa 82-96—SEQ ID NO: 1 | − |
| | #18 | Aa 87-101—SEQ ID NO: 1 | − |
| Pool 4 | #19 | Aa 92-106—SEQ ID NO: 1 | − |
| FABP7 | #20 | Aa 97-111—SEQ ID NO: 1 | − |
| | #21 | Aa 102-116—SEQ ID NO: 1 | + |
| | #22 | Aa 107-121—SEQ ID NO: 6 | − |
| | #23 | Aa 112-126—SEQ ID NO: 6 | − |
| | #24 | Aa 118-132—SEQ ID NO: 6 | + |
| Pool 5 | #25 | Aa 106-120—SEQ ID NO: 1 | − |
| FABP7 | #26 | Aa 111-125—SEQ ID NO: 1 | + |
| | #27 | Aa 116-130—SEQ ID NO: 1 | − |
| | #28 | Aa 121-135—SEQ ID NO: 1 | +++ |
| | #29 | Aa 126-140—SEQ ID NO: 1 | + |
| Pool 6 | #30 | Aa 131-145—SEQ ID NO: 1 | + |
| FABP7 | #31 | Aa 136-150—SEQ ID NO: 1 | − |
| | #32 | Aa 141-155—SEQ ID NO: 1 | − |
| | #33 | Aa 146-160—SEQ ID NO: 1 | ++ |
| | #34 | Aa 152-166—SEQ ID NO: 1 | + |
| Pool 1 | #1 | Aa 28-42—SEQ ID NO: 2 | − |
| PROK2 | #2 | Aa 33-47—SEQ ID NO: 2 | − |
| | #3 | Aa 38-52—SEQ ID NO: 2 | − |
| | #4 | Aa 43-57—SEQ ID NO: 2 | + |
| | #5 | Aa 48-62—SEQ ID NO: 2 | + |
| | #6 | Aa 53-67—SEQ ID NO: 2 | +++ |
| | #7 | Aa 58-72—SEQ ID NO: 2 | − |
| Pool 2 | #8 | Aa 63-77—SEQ ID NO: 2 | − |
| PROK2 | #9 | Aa 68-82—SEQ ID NO: 2 | + |
| | #10 | Aa 73-87—SEQ ID NO: 2 | + |
| | #11 | Aa 78-92—SEQ ID NO: 2 | − |
| | #12 | Aa 83-97—SEQ ID NO: 2 | − |
| | #13 | Aa 88-102—SEQ ID NO: 2 | − |
| | #14 | Aa 93-107—SEQ ID NO: 2 | + |
| Pool 3 | #15 | Aa 59-69 + 96-99—SEQ ID NO: 2 | − |
| PROK2 | #16 | Aa 64-69 + 96-104—SEQ ID NO: 2 | + |
| | #17 | Aa 69 + 96-109—SEQ ID NO: 2 | − |
| | #18 | Aa 100-114—SEQ ID NO: 2 | − |
| | #19 | Aa 105-119—SEQ ID NO: 2 | − |
| | #20 | Aa 110-124—SEQ ID NO: 2 | ++ |
| | #21 | Aa 115-129—SEQ ID NO: 2 | + |

### Example 7: Validation of Autoantigen Screening Using Recombinant Full-Length Antigens

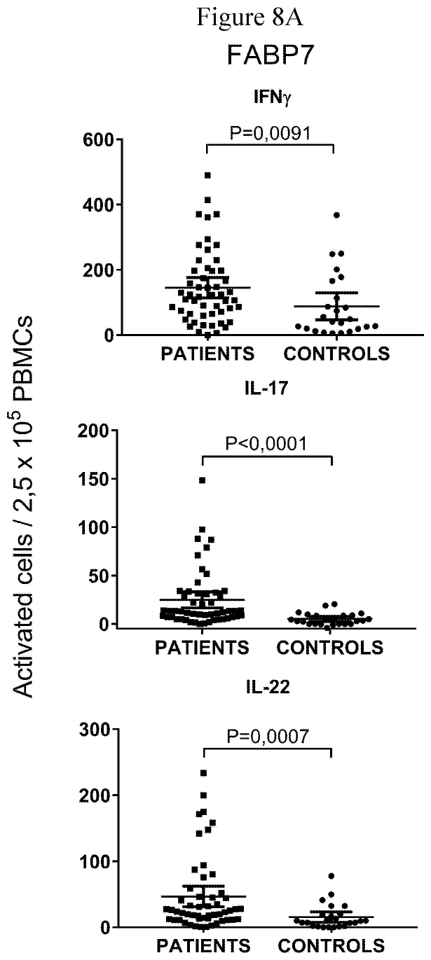

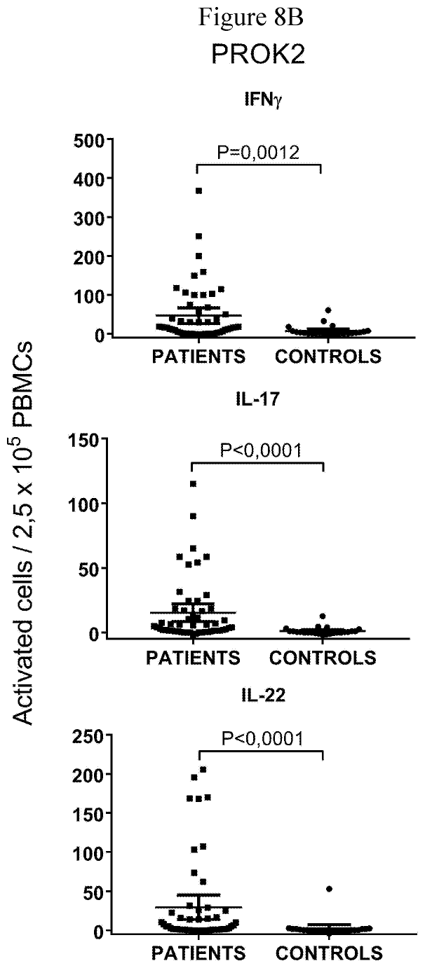

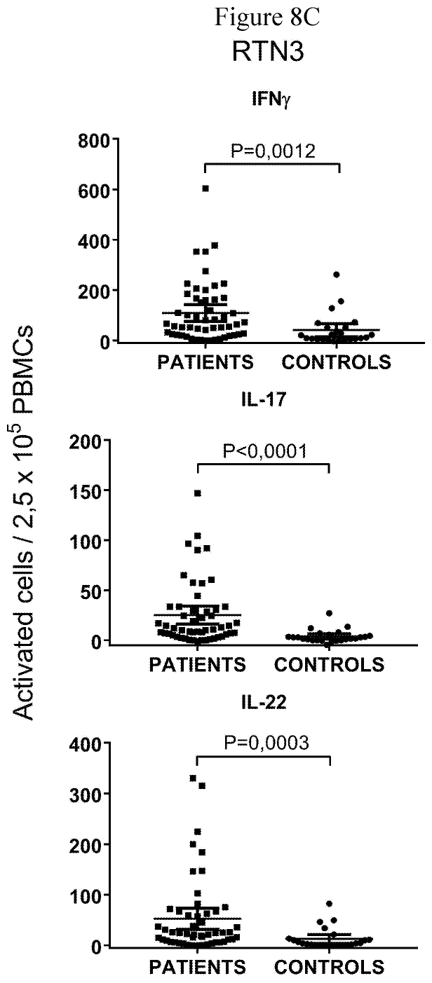

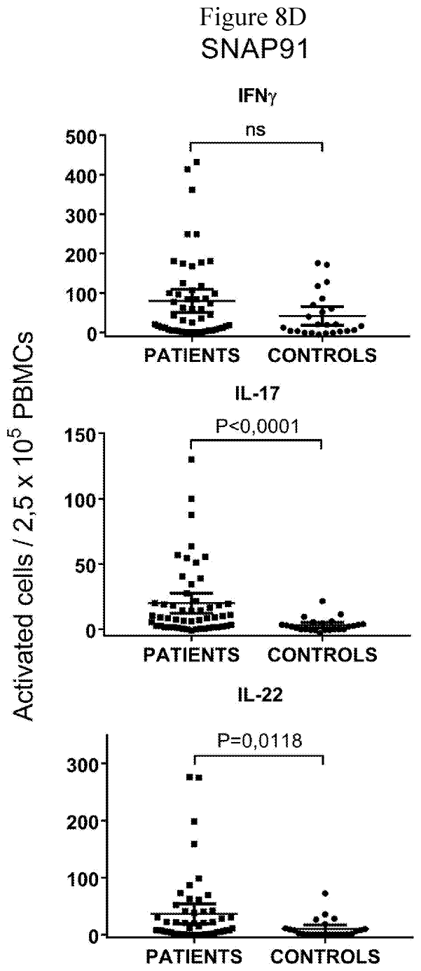

Recombinant full-length versions of the antigens FABP7 (SEQ ID NO: 1), PROK2 (SEQ ID NO: 2), RTN3 (SEQ ID NO: 3), SNAP91 (SEQ ID NO: 4) were produced in E. coli in house according to standard recombinant protein production protocols and purified via his-tag purification. 52 patients and 24 controls were tested for reactivity towards the antigens in an IFNγ/IL17/IL22 FluoroSpot assay as previously described. Similar as for the previously tested PrESTs, there was a significantly higher reactivity of multiple sclerosis patients PBMCs toward all four antigens for all cytokines analysed, apart from IFNγ for SNAP91 (See FIG. 8A-D).

### Example 8: Antigen Specific Immunotherapy

There are well described prior studies using administration, either subcutanous, intra- or transdermal, of previously known multiple sclerosis autoantigen peptide-epitopes in antigen-specific immunotherapy. For example, Walczak et al (Walczak A, Siger M, Szczepanik M, Selmaj K. Transdermal application of myelin peptides in multiple sclerosis treatment. JAMA Neurol. 2013) used a mix of known epitopes from the known autoantigens myelin basic protein (MBP), proteolipid protein (PLP) and myelin oligodendrocyte glycoprotein (MOG), applied transdermally over a 1-year period, with a significant but modest reduction in disease activity. Chataway et al (Cathaway J, Martin K, Barrell K, Sharrack B, Stolt P, Wraith D C. Effects of ATX-MS-1467 immunotherapy over 16 weeks in relapsing multiple sclerosis. Neurology 2018) used a mixture of four peptide epitopes from myelin basic protein (MBP), subcutaneously injected in increasing doses, over 4 weeks followed by biweekly doses of the highest dose over 16 weeks. Similarly, a significant positive but modest effect was seen. This indicates that the method of antigen specific immunotherapy via induction of T-cell tolerance towards the autoantigens is a working approach.

Based on the examples previously described in this document and known methods of epitope-mapping we will first identify the specific peptide-epitopes of FABP7, PROK2, RTN3 and SNAP 91. Patients will then, also as previously described, be screened for T-cell reactivity towards these autoantigens. Based on the screening, a patient-based mix of peptide-epitopes will be selected and then used for treatment according to established protocols for antigen specific immunotherapy. Treating with peptide-epitopes from more autoantigens than previously used, the efficacy can be expected to be greater whilst tolerability, due an individualised approach (and as such not including peptide-epitopes not relevant for the patient) stays the same.

Materials and Methods

Covalent Coupling of Proteins to Paramagnetic Beads Containing Free Carboxylic Acid Groups.

Dynabeads® MyOne™ Carboxylic Acid with 1 μm diameter (ThermoFischer Scientific) were used and the coupling procedure was carried out according to the manufacturers' protocol (Two-Step procedure using NHS (N-Hydroxysuccinimide) and EDC (ethyl carbodiimide)).

Beads were washed twice with MES-Buffer (25 mM MES (2-(N-morpholino)ethanesulfonic acid), pH 6). The carboxylic acid groups were then activated by adding 50 mg/ml NHS (N-Hydroxysuccinimide) and 50 mg/ml EDC (N-(3-Dimethylaminopropyl)-N′-ethylcarbodiimide) in MES-buffer to the beads and incubated for 30 minutes in room temperature. The beads were collected with a magnet and the supernatant was removed and the beads washed twice with MES-buffer. The protein was diluted in MES-buffer to a concentration of 1 mg/ml, total 100 ug and added to the beads and incubated for 1 h in room temperature. The beads were collected with a magnet and the supernatant was removed and saved for protein-concentration measurement. The non-reacted activated carboxylic acid groups were quenched with 50 mM Tris pH 7.4 for 15 minutes. The beads were then washed with PBS pH 7.4 and then stored in −80° C.

To measure the amount of protein coupled to the beads, a BCA protein assay kit (Pierce BCA Protein Assay Kit, ThermoFisher Scientific) was used to measure the protein concentration of the protein before coupling as well as in the supernatant after coupling. The BCA-assay was used according to the manufacturer's protocol.

Endotoxin Removal by Denaturing Washes.

Beads were coupled with a recombinant protein produced in E. coli. The protein-coupled beads were washed at several different denaturing conditions to ensure removal of endotoxin. For endotoxin removal, the beads were washed with 3 different wash-buffers, 2M NaOH pH 14.3, 0.5M L-Arginine and 0.1% Triton X100, all in sterile water at RT. The beads were suspended in the buffer and shaken for 4 min, collected with a magnet and the supernatant removed. This was repeated 5 times. The beads were then washed 5 times with sterile PBS to remove any remaining wash-buffer. The remaining endotoxin was measured using a monocyte reactivity assay (IL1B/IL6 FluoroSpot-assay, MABTECH, Sweden).

Patients

A total of 24 Patients with MS undergoing natalizumab treatment at the Neurological Clinic Karolinska University Hospital, Solna and Huddige, was asked to donate 80 ml venous blood in conjunction with their ordinary treatment visit. 6 of these patients were sampled again 6-12 months after the original sampling for use in further experiments (Example 5).

TABLE 5
Inclusion and exclusion criteria of MS-patients

| Inclusion Criteria | Exclusion Criteria |
|---|---|
| Confirmed MS-diagnosis | Active relapse at time of |
| | blood-sampling |
| Undergoing natalizumab | No and/or other current |
| treatment | treatment |
| Willing to participate in | Unwilling to participate |
| the study | in the study |
| Between 18 and 65 years | Other current serious |
| of age | illness |

Healthy controls, age and sex matched, were recruited amongst staff at the institution and clinic. The controls went through the same blood sampling procedure as the patients.

PBMCs were isolated from the venous blood samples (taken in BD Vacutainer EDTA-tubes) by Ficoll-Paque (GE Healthcare, Uppsala, Sweden) gradient centrifugation according to standard protocol. The cells were frozen in freezing medium (45% FCS, 45% RPMI, 10% DMSO) and stored in −150° C.

A second, larger, cohort was collected in order to test the full length antigens. In this cohort, 52 patients and 24 healthy controls were included. The inclusion/exclusion criteria (table 5) as well as the sampling and PBMCs isolation were the same.

Candidate Antigen Library

The antigens used was acquired from the Royal Institute of Technology (KTH, Sweden) and the Swedish Human Protein Atlas project (Uhlen M, Fagerberg L, Hallstrom B M, Lindskog C, Oksvold P, Mardinoglu A, et al. Proteomics. Tissue-based map of the human proteome. Science. 2015; 347(6220):1260419). They consist of protein epitope signature tags (PrESTs), short recombinant 10-12 kDa peptides representing unique parts of human proteins. The PrESTs have been produced in Escherichia coli and purified via a tag. The PrESTs for this project were selected by the following criteria: 1) Proteins assumed to be involved in MS according to published data. 2) Major structural proteins of the myelin sheets. 3) Proteins of interest, selected by communication with experts in the field. 4) Highly expressed CNS specific proteins previously not associated with disease. Altogether 125 PrESTs from 70 different proteins were used in this trial.

Production of Full-Length Antigens

The antigens (FABP7, PROK2, RTN3 and SNAP91) picked out from the screening of PrESTs described in Example 1 and 2 were selected for further testing on the full-length versions of the protein. The full-length antigens were produced by transforming plasmids encoding the antigen and a histamine tag into Escherichia coli. After growth, the bacteria were lysed and the protein purified from the supernatant of the bacterial lysis via 6×histidine-tag purification on an immobilized metal affinity chromatography-column. The antigens were subsequently coupled to beads and endotoxin-washed as previously described.

FluoroSpot T-Cell Activation Assay

The FluoroSpot assay was performed under sterile conditions in a cell culture hood. The cells were thawed in a water bath at 37° C. and washed with cRPMI (RPMI 1640 medium, Sigma Aldrich, containing 10% fetal calf serum, 1% 200 mM L-Glutamine, 1% 10,000 U/ml Penicillin-10 mg/ml Streptomycin). The cells were counted manually using a light-microscope (Nikon TMS-F, Nikon, Japan) and subsequently diluted in cRPMI to a concentration of 2.5×10⁶ cells/ml. The FluoroSpot plate (Human IFNγ/IL-22/IL-17A FluoroSpot kit, pre-coated, Mabtech, Sweden) was washed with PBS and then blocked with cRPMI for 30min at room temperature. The blocking cRPMI was then discarded and 100p.I fresh cRPMI was added to each well of the FluoroSpot plate. Antigen (3×10\hat{ }6 beads) was added to each well in duplicates according to a specific layout. In accordance to the manufacturer's protocol, anti-CD3 was used as a positive control. Both uncoupled beads and media without stimuli was used as negative controls. PBMCs (250,000 cells) in 100 μl cRPMI were added to each well (125,000 cells for the anti-CD3). The plates were placed in an incubator (37° C. humidified, 5% CO2) for 44 hours. The development of spots was prepared according to the manufacturer's protocol.

Overlapping Peptides

Continuous overlapping peptides (15 amino acids in length with a 10 amino acid overlap) spanning the whole of FABP7 isoforms 1 and 2, and PROK2 were purchased in lyophilised form from a commercial vendor and were subsequently suspended in 100% dimethyl sulfoxide (DMSO) to a concentration of 50-100 mg/ml. They were then further diluted in sterile PBS to a concentration of 5 mg/ml. They were pooled into 6 fractions for FABP7 and 3 fractions for PROK2, containing 5-7 peptides each. Each pool contained located next to each other on the full-length protein. Cells from 6 MS-patients were tested against these in a FluoroSpot assay according to previous described protocol. The final concentration of each peptide in the cell culture well were 5 μg/ml.

Antigen-Specific Immunotherapy

A clinical trial will be made to assess the safety, tolerability and efficacy of an antigen specific immunotherapy targeting the identified autoantigens FABP7, PROK2, RTN3, and SNAP91. Firstly, the immuno-dominant epitopes from each antigen will be identified as previously explained in Example 6. Secondly, the study participants (multiple sclerosis patients) will be screened for T-cell activity towards the autoantigens using the methods described in Example 7. Patients with reactivity towards the autoantigens will be eligible for inclusion in the trial. The treatment can either consist of a mix of one or several immuno-dominant peptide epitopes from each autoantigen or a mix of one or several immuno-dominant peptide epitopes from only the autoantigens the patients reacted to in the pre-inclusion screening.

Treatment protocol will be based on previously published successful antigen-specific immunotherapy protocols (CathawayJ, Martin K, Barrell K, Sharrack B, Stolt P, Wraith D C. Effects of ATX-MS-1467 immunotherapy over 16 weeks in relapsing multiple sclerosis. Neurology 2018) (Walczak A, Siger M, Szczepanik M, Selmaj K. Transdermal application of myelin peptides in multiple sclerosis treatment. JAMA Neurol. 2013). In one alternative protocol, the treatment will consist of weekly/biweekly subcutaneous or intradermal injection of the peptide-epitope mix, starting with a low dose followed by an up-dosing period until the desired higher dose is reached. This will be followed by a period of weekly/biweekly injections of the higher dose for a limited period. Alternatively, the peptide-epitopes will be administered under a similar scheme but either dermally, sublingual or orally. Safety and tolerability will be continuously evaluated while efficacy will be evaluated after full treatment. Endpoints will consist of efficacy parameters such as a combination of magnetic resonance imaging-based evaluation of the number and volume of lesions and clinical variables including expanded disability status score (EDSS) and time to first relapse or frequency of relapses.

1-91. (canceled)

92. A method of treatment for multiple sclerosis (MS) in a MS patient exhibiting T-cell autoreactivity against an endogenous epitope corresponding to a specific T-cell epitope comprised in the amino-acid sequence of SEQ ID NO: 5,
comprising administering to the patient in a tolerogenic manner a composition, thus inducing T-cell tolerance towards said T-cell epitope in the patient,
the composition comprising:
a) a therapeutic T-cell epitope comprising a sequence of n consecutive amino acid residues being:
a. identical to a sub-sequence of SEQ ID NO: 5; or
b. differing from a sub-sequence of SEQ ID NO: 5 by no more than m residue substitutions, deletions and/or insertions;
wherein n is at least 8, and m is 0, 1 or 2;
b) a nucleic acid encoding a therapeutic T-cell epitope comprising a sequence of n consecutive amino acid residues being:
a. identical to a sub-sequence of SEQ ID NO: 5; or
b. differing from a sub-sequence of SEQ ID NO: 5 by no more than m residue substitutions, deletions and/or insertions;
wherein n is at least 8, and m is 0, 1 or 2; or
c) an antigen-presenting cell exposed ex vivo to a therapeutic T-cell epitope comprising a sequence of n consecutive amino acid residues being:
a. identical to a sub-sequence of SEQ ID NO: 5; or
b. differing from a sub-sequence of SEQ ID NO: 5 by no more than m residue substitutions, deletions and/or insertions;
wherein n is at least 8, and m is 0, 1 or 2.

93. The method of treatment according to claim 92, comprising determining the patient's T-cell autoreactivity against a T-cell epitope comprised in the amino-acid sequence of SEQ ID NO: 5.

94. The method of treatment according to claim 92, wherein the sub-sequence is comprised in residues 1-166 of SEQ ID NO: 5.

95. The method of treatment according to claim 92, wherein the sub-sequence is comprised in residues 167-295 of SEQ ID NO: 5.

96. The method of treatment according to claim 92, wherein the sub-sequence is comprised in residues 296-1327 of SEQ ID NO: 5.

97. The method of treatment according to claim 92, wherein the sub-sequence is comprised in residues 1328-2234 of SEQ ID NO: 5.

98. The method of treatment according to claim 92, wherein the sub-sequence is comprised in residues 2235-2250 of SEQ ID NO: 5.

99. The method of treatment according to claim 92, wherein the sub-sequence is comprised in residues 1-2234 of SEQ ID NO: 5.

100. The method of treatment according to claim 92, wherein the sub-sequence is comprised in any one of the sequences of peptides in table 4.

101. The method of treatment according to claim 92, wherein n is at least 11.

102. The method of treatment according to claim 92, wherein n is at least 15.

103. The method of treatment according to claim 92, wherein the composition comprises the therapeutic T-cell epitope coupled to solid carrier, such as a biocompatible polymer, a particle or a cell.

104. The method of treatment according to claim 92, comprising selecting the therapeutic T-cell epitope such that it corresponds to an epitope to which the patient exhibits T-cell autoreactivity.

105. A method for determining the degree of multiple sclerosis (MS) related autoimmunity in a test subject, comprising:
a. providing a test sample derived from the test subject comprising viable T-cells;
b. quantitating antigen-specific activation of the T-cells of the test sample in vitro in response to a test antigen comprising a T-cell epitope, wherein said T-cell epitope comprises an amino-acid sequence of n consecutive residues being:
i. identical to a sub-sequence of SEQ ID NO: 5; or
ii. differing from a sub-sequence of SEQ ID NO: 5 by no more than m residue substitutions, deletions and/or insertions;
wherein n is at least 8, and m is 0, 1 or 2; and
c. comparing the quantitated antigen-specific activation to a relevant reference to determine the degree of MS-related autoimmunity in the test subject.

106. The method according to claim 105, further comprising:
a. providing a viable antigen-presenting cell;
b. contacting the test antigen with the antigen-presenting cell;
c. contacting in vitro the test sample with the antigen-presenting cell contacted with the test antigen under conditions allowing antigen-specific activation of T-cells in response to an antigen presented by an antigen-presenting cell; and
d. quantitating antigen-specific T-cell activation in the test sample.

107. The method according to claim 106, wherein the method comprises providing the test antigen tightly associated to a phagocytable particle.

108. The method according to claim 107, wherein quantitating the antigen-specific T-cell activation in the test sample comprises determining the T-cell response by measuring secretion of IFN-γ, IL-17 or IL-22.

109. The method according to claim 105, wherein the quantitation of antigen-specific T-cell activation comprises the steps of:
a. providing a phagocytable particle, having the test antigen tightly associated thereto, wherein the particle with the associated test antigen has been subjected to a denaturing wash resulting in an endotoxin level low enough to not interfere with the subsequent steps;
b. providing a viable antigen-presenting cell;
c. contacting the washed particle with the antigen-presenting cell under conditions allowing phagocytosis of the particle by the antigen-presenting cell;
d. providing the test sample to be assayed comprising viable T-cells;
e. contacting in vitro the test sample with the antigen-presenting cell contacted with the particle under conditions allowing antigen-specific activation of T-cells in response to an antigen presented by an antigen-presenting cell; and
f. quantitating antigen-specific T-cell activation in the test sample.

110. The method according to claim 109, wherein quantitating the antigen-specific T-cell activation in the test sample comprises determining the T-cell response by measuring secretion of IFN-γ, IL-17 or IL-22.

111. A composition comprising a therapeutic T-cell epitope comprising a sequence of n consecutive amino acid residues being:
a. identical to a sub-sequence of SEQ ID NO: 5; or
b. differing from a sub-sequence of SEQ ID NO: 5 by no more than m residue substitutions, deletions and/or insertions;
wherein n is at least 8, and m is 0, 1 or 2.


SEQUENCE LISTING (6 sequences)

SEQ ID NO: 1 - protein, 166 aa, Homo sapiens
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMVSNDNSPFFLVFFSSPHTSHLLPSSSLLLPFFLLPSFFNNTSLARFFNYM

SEQ ID NO: 2 - protein, 129 aa, Homo sapiens
MRSLCCAPLLLLLLLPPLLLTPRAGDAAVITGACDKDSQCGGGMCCAVSIWVKSIRICTPMGKLGDSCHPLTRKNNFGNGRQERRKRKRSKRKKEVPFFGRRMHHTCPCLPGLACLRTSFNRFICLAQK

SEQ ID NO: 3 - protein, 1032 aa, Homo sapiens
MAEPSAATQSHSISSSSFGAEPSAPGGGGSPGACPALGTKSCSSSCADSFVSSSSSQPVSLFSTSQEGLSSLCSDEPSSEIMTSSFLSSSEIHNTGLTILHGEKSHVLGSQPILAKEGKDHLDLLDMKKMEKPQGTSNNVSDSSVSLAAGVHCDRPSIPASFPEHPAFLSKKIGQVEEQIDKETKNPNGVSSREAKTALDADDRFTLLTAQKPPTEYSKVEGIYTYSLSPSKVSGDDVIEKDSPESPFEVIIDKAAFDKEFKDSYKESTDDFGSWSVHTDKESSEDISETNDKLFPLRNKEAGRYPMSALLSRQFSHTNAALEEVSRCVNDMHNFTNEILTWDLVPQVKQQTDKSSDCITKTTGLDMSEYNSEIPVVNLKTSTHQKTPVCSIDGSTPITKSTGDWAEASLQQENAITGKPVPDSLNSTKEFSIKGVQGNMQKQDDTLAELPGSPPEKCDSLGSGVATVKVVLPDDHLKDEMDWQSSALGEITEADSSGESDDTVIEDITADTSFENNKIQAEKPVSIPSAVVKTGEREIKEIPSCEREEKTSKNFEELVSDSELHQDQPDILGRSPASEAACSKVPDTNVSLEDVSEVAPEKPITTENPKLPSTVSPNVFNETEFSLNVTTSAYLESLHGKNVKHIDDSSPEDLIAAFTETRDKGIVDSERNAFKAISEKMTDFKTTPPVEVLHENESGGSEIKDIGSKYSEQSKETNGSEPLGVFPTQGTPVASLDLEQEQLTIKALKELGERQVEKSTSAQRDAELPSEEVLKQTFTFAPESWPQRSYDILERNVKNGSDLGISQKPITIRETTRVDAVSSLSKTELVKKHVLARLLTDFSVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 4 - protein, 907 aa, Homo sapiens
MSGQTLTDRIAAAQYSVTGSAVARAVCKATTHEVMGPKKKHLDYLIQATNETNVNIPQMADTLFERATNSSWVVVFKALVTTHHLMVHGNERFIQYLASRNTLFNLSNFLDKSGSHGYDMSTFIRRYSRYLNEKAFSYRQMAFDFARVKKGADGVMRTMAPEKLLKSMPILQGQIDALLEFDVHPNELTNGVINAAFMLLFKDLIKLFACYNDGVINLLEKFFEMKKGQCKDALEIYKRFLTRMTRVSEFLKVAEQVGIDKGDIPDLTQAPSSLMETLEQHLNTLEGKKPGNNEGSGAPSPLSKSSPATTVTSPNSTPAKTIDTSPPVDLFATASAAVPVSTSKPSSDLLDLQPDFSSGGAAAAAAPAPPPPAGGATAWGDLLGEDSLAALSSVPSEAQISDPFAPEPTPPTTTAEIATASASASTTTTVTAVTAEVDLFGDAFAASPGEAPAASEGAAAPATPTPVAAALDACSGNDPFAPSEGSAEAAPELDLFAMKPPETSVPVVTPTASTAPPVPATAPSPAPAVAAAAAATTAATAAATTTTTTSAATATTAPPALDIFGDLFESTPEVAAAPKPDAAPSIDLFSTDAFSSPPQGASPVPESSLTADLLSVDAFAAPSPATTASPAKVDSSGVIDLFGDAFGSSASEPQPASQAASSSSASADLLAGFGGSFMAPSPSPVTPAQNNLLQPNFEAAFGTTPSTSSSSSFDPSVFDGLGDLLMPTMAPAGQPAPVSMVPPSPAMAASKALGSDLDSSLASLVGNLGISGTTTKKGDLQWNAGEKKLTGGANWQPKVAPATWSAGVPPSAPLQGAVPPTSSVPPVAGAPSVGQPGAGFGMPPAGTGMPMMPQQPVMFAQPMMRPPFGAAAVPGTQLSPSPTPASQSPKKPPAKDPLADLNIKDFL

SEQ ID NO: 5 - protein, 2250 aa, Artificial
  Combined sequence of human proteins FABP7, PROK2, RTN3 and SNAP91.
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMVSNDNSPFFLVFFSSPHTSHLLPSSSLLLPFFLLPSFFNNTSLARFFNYMMRSLCCAPLLLLLLLPPLLLTPRAGDAAVITGACDKDSQCGGGMCCAVSIWVKSIRICTPMGKLGDSCHPLTRKNNFGNGRQERRKRKRSKRKKEVPFFGRRMHHTCPCLPGLACLRTSFNRFICLAQKMAEPSAATQSHSISSSSFGAEPSAPGGGGSPGACPALGTKSCSSSCADSFVSSSSSQPVSLFSTSQEGLSSLCSDEPSSEIMTSSFLSSSEIHNTGLTILHGEKSHVLGSQPILAKEGKDHLDLLDMKKMEKPQGTSNNVSDSSVSLAAGVHCDRPSIPASFPEHPAFLSKKIGQVEEQIDKETKNPNGVSSREAKTALDADDRFTLLTAQKPPTEYSKVEGIYTYSLSPSKVSGDDVIEKDSPESPFEVIIDKAAFDKEFKDSYKESTDDFGSWSVHTDKESSEDISETNDKLFPLRNKEAGRYPMSALLSRQFSHTNAALEEVSRCVNDMHNFTNEILTWDLVPQVKQQTDKSSDCITKTTGLDMSEYNSEIPVVNLKTSTHQKTPVCSIDGSTPITKSTGDWAEASLQQENAITGKPVPDSLNSTKEFSIKGVQGNMQKQDDTLAELPGSPPEKCDSLGSGVATVKVVLPDDHLKDEMDWQSSALGEITEADSSGESDDTVIEDITADTSFENNKIQAEKPVSIPSAVVKTGEREIKEIPSCEREEKTSKNFEELVSDSELHQDQPDILGRSPASEAACSKVPDTNVSLEDVSEVAPEKPITTENPKLPSTVSPNVFNETEFSLNVTTSAYLESLHGKNVKHIDDSSPEDLIAAFTETRDKGIVDSERNAFKAISEKMTDFKTTPPVEVLHENESGGSEIKDIGSKYSEQSKETNGSEPLGVFPTQGTPVASLDLEQEQLTIKALKELGERQVEKSTSAQRDAELPSEEVLKQTFTFAPESWPQRSYDILERNVKNGSDLGISQKPITIRETTRVDAVSSLSKTELVKKHVLARLLTDFSVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAEMSGQTLTDRIAAAQYSVTGSAVARAVCKATTHEVMGPKKKHLDYLIQATNETNVNIPQMADTLFERATNSSWVVVFKALVTTHHLMVHGNERFIQYLASRNTLFNLSNFLDKSGSHGYDMSTFIRRYSRYLNEKAFSYRQMAFDFARVKKGADGVMRTMAPEKLLKSMPILQGQIDALLEFDVHPNELTNGVINAAFMLLFKDLIKLFACYNDGVINLLEKFFEMKKGQCKDALEIYKRFLTRMTRVSEFLKVAEQVGIDKGDIPDLTQAPSSLMETLEQHLNTLEGKKPGNNEGSGAPSPLSKSSPATTVTSPNSTPAKTIDTSPPVDLFATASAAVPVSTSKPSSDLLDLQPDFSSGGAAAAAAPAPPPPAGGATAWGDLLGEDSLAALSSVPSEAQISDPFAPEPTPPTTTAEIATASASASTTTTVTAVTAEVDLFGDAFAASPGEAPAASEGAAAPATPTPVAAALDACSGNDPFAPSEGSAEAAPELDLFAMKPPETSVPVVTPTASTAPPVPATAPSPAPAVAAAAAATTAATAAATTTTTTSAATATTAPPALDIFGDLFESTPEVAAAPKPDAAPSIDLFSTDAFSSPPQGASPVPESSLTADLLSVDAFAAPSPATTASPAKVDSSGVIDLFGDAFGSSASEPQPASQAASSSSASADLLAGFGGSFMAPSPSPVTPAQNNLLQPNFEAAFGTTPSTSSSSSFDPSVFDGLGDLLMPTMAPAGQPAPVSMVPPSPAMAASKALGSDLDSSLASLVGNLGISGTTTKKGDLQWNAGEKKLTGGANWQPKVAPATWSAGVPPSAPLQGAVPPTSSVPPVAGAPSVGQPGAGFGMPPAGTGMPMMPQQPVMFAQPMMRPPFGAAAVPGTQLSPSPTPASQSPKKPPAKDPLADLNIKDFLTLTFGDVVAVRHYEKA

SEQ ID NO: 6 - protein, 132 aa, homo sapiens
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA